(19) United States
(12) Patent Application Publication
(10) Pub. No.: US 2018/0001591 A1
(43) Pub. Date: Jan. 4, 2018

(54) HIGH PERFORMANCE NONWOVEN STRUCTURE

(71) Applicant: Georgia-Pacific Nonwovens LLC, Atlanta, GA (US)

(72) Inventors: Jacek K. Dutkiewicz, Cordova, TN (US); Brian Fong, Lakeland, TN (US)

(73) Assignee: Georgia-Pacific Nonwovens LLC, Atlanta, GA (US)

(21) Appl. No.: 15/542,822

(22) PCT Filed: Jan. 12, 2016
(86) PCT No.: PCT/US16/13117

§ 371 Date: Jul. 11, 2017

Related U.S. Application Data
(60) Provisional application No. 62/102,404, filed on Jan. 12, 2015.
(60) Provisional application No. 62/142,660, filed on Apr. 3, 2015.

(51) Int. Cl. B32B 5/02; B32B 5/26; A61L 15/34; A61F 13/537; A61F 13/511

(52) U.S. Cl. CPC B32B 5/022; A61F 13/53713; A61F 13/53717; A61F 13/5116; A61L 15/34; B32B 5/26; A61F 2013/51178; B32B 2262/04; B32B 2262/062; B32B 2262/12; B32B 2555/00; B32B 2555/02; B32B 2262/02

(57) ABSTRACT

The presently disclosed subject matter relates to multi-layer nonwoven materials and their use in absorbent articles. More particularly, the presently disclosed subject matter relates to layered structures that have high absorbency performance while having less absorbent mass than other commercially available materials.

### 1. CROSS-REFERENCE TO RELATED APPLICATIONS

This application claims priority to U.S. Provisional Application No. 62/102,404, filed Jan. 12, 2015, and U.S. Provisional Application No. 62/142,660, filed Apr. 3, 2015, the contents of which are hereby incorporated by reference in their entireties.

### 2. FIELD OF THE INVENTION

The presently disclosed subject matter relates to new nonwoven materials and their use in articles including diapers and incontinence products, feminine hygiene products, and other consumer products such as cleaning products. More particularly, the presently disclosed subject matter relates to structures containing low absorbent mass with an improved fluid acquisition and dryness profile as well as added retention properties.

### 3. BACKGROUND OF THE INVENTION

Nonwoven structures are important in a wide range of consumer products, such as absorbent articles including baby diapers, adult incontinence products, sanitary napkins, cleaning products, and the like. In certain nonwoven articles, there is often an absorbent core to receive and retain body liquids. The absorbent core is usually sandwiched between a liquid pervious topsheet, whose function is to allow the passage of fluid to the core and a liquid impervious backsheet whose function is to contain the fluid and to prevent it from passing through the absorbent article to the garment of the wearer of the absorbent article.

In the conventional multi-layer absorbent structure or system having an acquisition layer, a distribution layer and a storage layer, the acquisition layer acquires the liquid insult and quickly transmits it by capillary action away from the skin of the wearer (in the Z-direction). Next, the fluid encounters the distribution layer. The distribution layer is typically of a higher density material, and causes the liquid to migrate away from the skin of the wearer (in the Z-direction) and also laterally across the structure (in the X-Y directions). Finally, the liquid migrates into the storage layer. The storage layer generally includes high density cellulose fibers and SAP particles. The liquid is absorbed by the storage layer and especially the SAP particles contained therein.

In other conventional multi-layer absorbent structures or systems having an acquisition layer and a storage layer, the acquisition layer acquires the liquid insult and distributes the liquid away from the skin of the wearer. The liquid migrates and is absorbed into the storage layer.

In recent years, market demand for an increasingly thinner and more comfortable absorbent article has increased. Such an article may be obtained by decreasing the thickness of the core, by increasing the amount of SAP particles, and by calendaring or pressing the core to reduce caliper and hence, increase density. However, higher density cores do not absorb liquid as rapidly as lower density cores because densification of the core results in a smaller effective pore size. Therefore, to maintain suitable liquid absorption, it is necessary to provide a low-density layer having a larger pore size above the high-density absorbent core to increase the rate of uptake of liquid discharged onto the absorbent article. The low-density layer is typically referred to as an acquisition layer.

Pliability and softness of the absorbent core are necessary to ensure that the absorbent core can easily conform itself to the shape of the human body or to the shape of a component (for example another absorbent ply) of the absorbent article adjacent to it. This in turn prevents the formation of gaps and channels between the absorbent article and the human body or between various parts of the absorbent article, which might otherwise cause undesired leaks in the absorbent article. Integrity of the absorbent core is necessary to ensure that the absorbent core does not deform and exhibit discontinuities during its use by a consumer. Such deformations and discontinuities can lead to a decrease in overall absorbency and capacity, and an increase in undesired leakages. Prior absorbent structures have been deficient in one or more of pliability, integrity, profiled absorbency and capacity.

Thus, there remains a need for a nonwoven material that has enough absorbent capacity for its intended use, and yet be conformable with the desired dryness profile. The disclosed subject matter addresses these needs.

### 4. SUMMARY

The presently disclosed subject matter provides for an absorbent structure with a multi-layer nonwoven material containing specific layered constructions, which advantageously achieve high overall absorbency performance with less absorbent mass, and provide better fluid acquisition and dryness characteristics at comparable basis weights.

The presently disclosed subject matter provides for a multi-layer nonwoven material having at least two layers, at least three layers, at least four layers, at least five layers, or at least six layers.

In certain embodiments, the disclosed subject matter provides for a multi-layer nonwoven acquisition material having a first outer layer containing synthetic fibers and having a basis weight from about 10 gsm to about 50 gsm. A second outer layer can contain cellulose fibers and binder and have a basis weight from about 10 gsm to about 100 gsm. The multi-layer nonwoven acquisition material can have a caliper from about 0.5 mm to about 4 mm, a basis weight from about 10 gsm to about 200 gsm, and a tensile strength at peak load of greater than about 400 G/in.

In particular embodiments, the first outer layer can further include binder. The synthetic fibers of the first outer layer can be bicomponent fibers. The multi-layer nonwoven acquisition material can have additional layers. For example, the multi-layer nonwoven acquisition material can have a first intermediate layer containing bicomponent fibers. In certain embodiments, the multi-layer nonwoven acquisition material can have a second intermediate layer containing cellulose fibers and bicomponent fibers. In certain embodiments, the multi-layer nonwoven acquisition material can further include an absorbent core. In certain embodiments, the multi-layer nonwoven acquisition material can be part of an absorbent composite.

In other embodiments, the disclosed subject matter provides for a multi-layer nonwoven acquisition material having a first outer layer containing synthetic fibers and having a basis weight from about 10 gsm to about 50 gsm. A second outer layer can contain synthetic filaments. The multi-layer nonwoven acquisition material can have a caliper from about 0.5 mm to about 4 mm and a basis weight from about 10 gsm to about 200 gsm.

In particular embodiments, the first outer layer can further include binder. The synthetic fibers of the first outer layer can be bicomponent fibers. The multi-layer nonwoven acquisition material can have additional layers. For example, the multi-layer nonwoven acquisition material can have a first intermediate layer containing bicomponent fibers. In certain embodiments, the multi-layer nonwoven acquisition material can further include an absorbent core. In certain embodiments, the multi-layer nonwoven acquisition material can be part of an absorbent composite.

In certain embodiments, the disclosed subject matter provides for a multi-layer nonwoven material having an outer layer containing synthetic fibers and an absorbent core. The outer layer can have a basis weight from about 10 gsm to about 50 gsm. The multi-layer nonwoven material can have a caliper from about 1 mm to about 8 mm and a basis weight from about 100 gsm to about 600 gsm.

In particular embodiments, the outer layer can further include binder. The synthetic fibers of the outer layer can be bicomponent fibers. In certain embodiments, the absorbent core can have a first layer containing cellulose fibers, a second layer containing SAP, a third layer containing cellulose fibers, a fourth layer containing SAP, and a fifth layer containing cellulose fibers. One or more of the first layer, third layer, and fifth layer of the absorbent core can further include bicomponent fibers. In certain embodiments, the fifth layer of the absorbent core can further include binder. In certain embodiments, the multi-layer nonwoven material can be part of an absorbent composite.

### 5. BRIEF DESCRIPTION OF THE DRAWINGS

FIG. 1 provides an illustration of the three-layer acquisition material of Example 1. Note that in FIG. 1 and subsequent Figures, rows correspond to layers of the material and provide the composition of each layer.

FIG. 2 provides an illustration of the acquisition times of the two materials of Example 1 after each of three insults. A first material (“with bico”) contained bicomponent fibers and a second material (“without bico”) did not.

FIG. 3 provides an illustration of the rewet results of the two acquisition materials of Example 1. A first material (“with bico”) contained bicomponent fibers and a second material (“without bico”) did not. The rewet results are provided as a weight (g) of liquid that was released from the materials.

FIG. 4 depicts the three-layer acquisition material of Example 2.

FIG. 5 provides an illustration of the runoff percentage (%) of insult for the two acquisition materials of Example 2.

FIG. 6 provides an illustration of the acquisition times of the two control materials in Example 3 after each of three insults.

FIGS. 7A-7J provide illustrations of Structures 4A-4J, respectively, of Example 4.

FIG. 8 provides an illustration of the acquisition times of Structures 4A-4J of Example 4 after each of three insults.

FIGS. 9A-9C provide illustrations of Structures 5A-5C, respectively, of Example 5.

FIG. 10 provides an illustration of the acquisition times of Structures 5A-5C of Example 5 after each of three insults.

FIGS. 11A-11C provide illustrations of Structures 6A-6C, respectively, of Example 6.

FIG. 12 provides an illustration of the acquisition times of Structures 6A-6C of Example 6 after each of three insults.

FIG. 13 provides an illustration of the rewet results of Structures 6A-6C of Example 6. The rewet results are provided as a weight (g) of liquid that was released from the materials.

FIGS. 14A-14B provide illustrations of Structures 7A-7B, respectively, of Example 7.

FIG. 15 provides an illustration of the acquisition times Structures 7A-7B of Example 7 after each of three insults.

FIGS. 16A-16B provide illustrations of Structures 8A-8B, respectively, of Example 8.

FIG. 17 provides an illustration of the acquisition times of Structures 8A-8B of Example 8 after each of three insults.

FIGS. 18A-18C provide illustrations of Structures 9A-9C, respectively, of Example 9.

FIG. 19 provides an illustration of the acquisition times of Structures 10A-10B of Example 10 after each of three insults. Results corresponding to Vicell 6609 are provided for comparison.

FIG. 20 provides an illustration of Structure 11A of Example 11.

FIG. 21 provides an illustration of the testing apparatus used in Examples 11 and 12.

FIG. 22 provides an illustration of the acquisition times of the materials of Example 11 after each of three insults. The first material contained a high-loft acquisition layer (“High-Loft”) and the second material contained Structure 11A.

FIG. 23 provides an illustration of the rewet results of the materials in Example 11. The first material contained a high-loft acquisition layer (“High-Loft”) and the second material contained Structure 11A. The rewet results are provided as a weight (grams) of liquid that was released from the materials.

FIG. 24 provides an illustration of Structure 12A of Example 12.

FIG. 25 provides an illustration of the acquisition times of the materials of Example 12 after each of three insults. The first material contained a high-loft acquisition layer (“High-Loft”) and the second material contained Structure 12A.

FIG. 26 provides an illustration of the rewet results of the Structures of Example 12. The first material contained a high-loft acquisition layer (“High-Loft”) and the second material contained Structure 12A. The rewet results are provided as a weight (grams) of liquid that was released from the materials.

FIG. 27 provides an illustration of the acquisition times of the materials of Example 13 after each of three insults. Structure 13A of Example 13 is compared to 175 MBS3A, a commercially available absorbent core material.

FIG. 28 provides an illustration of Structure 14A of Example 14.

FIG. 29 provides an illustration of the acquisition times of two materials of Example 14 after each of three insults. A commercially available product (“Product A”) is compared to a modified product containing Structure 14A of Example 14.

FIG. 30 provides an illustration of the humidity sensation of two materials of Example 14. A commercially available product (“Product A”) is compared to a modified product containing Structure 14A of Example 14. The humidity sensation is provided as a weight (mg) of liquid that was released from the materials.

FIG. 31 provides an illustration of the acquisition times of two materials of Example 14 after each of three insults. A commercially available product (“Product B”) is compared to a modified product containing Structure 14A of Example 14.

FIG. 32 provides an illustration of the humidity sensation of two materials of Example 14. A commercially available product (“Product B”) is compared to a modified product containing Structure 14A of Example 14. The humidity sensation is provided as a weight (mg) of liquid that was released from the materials.

FIG. 33 provides an illustration of the acquisition times of Structures 15A-15C of Example 15 after each of three insults. The acquisition times of Vicell 6609/Core are provided for comparison.

FIGS. 34A-34B provide illustrations of Structures 17A-17B, respectively, of Example 17.

FIG. 35 provides an illustration of the acquisition times of two materials of Example 17 after each of three insults. A commercially available product (“Product A”) is compared to a modified product containing Structure 17A of Example 17.

FIG. 36 provides an illustration of the humidity sensation of two Structures in Example 17. A commercially available product (“Product A”) is compared to a modified product containing Structure 17A of Example 17. The humidity sensation is provided as a weight (mg) of liquid that was released from the materials.

FIG. 37 provides an illustration of the acquisition times of two Structures in Example 17. A commercially available product (“Product B”) is compared to a modified product containing Structure 17B of Example 17.

FIG. 38 provides an illustration of the humidity sensation of two Structures in Example 17. A commercially available product (“Product B”) is compared to a modified product containing Structure 17B of Example 17. The humidity sensation is provided as a weight (mg) of liquid that was released from the materials.

### 6. DETAILED DESCRIPTION

The presently disclosed subject matter provides multi-layer nonwoven materials for use in absorbent articles. The presently disclosed subject matter also provides methods for making such materials. These and other aspects of the disclosed subject matter are discussed more in the detailed description and examples.

### Definitions

The terms used in this specification generally have their ordinary meanings in the art, within the context of this subject matter and in the specific context where each term is used. Certain terms are defined below to provide additional guidance in describing the compositions and methods of the disclosed subject matter and how to make and use them.

As used herein, a “nonwoven” refers to a class of material, including but not limited to textiles or plastics. Nonwovens are sheet or web structures made of fiber, filaments, molten plastic, or plastic films bonded together mechanically, thermally, or chemically. A nonwoven is a fabric made directly from a web of fiber, without the yarn preparation necessary for weaving or knitting. In a nonwoven, the assembly of fibers is held together by one or more of the following: (1) by mechanical interlocking in a random web or mat; (2) by fusing of the fibers, as in the case of thermoplastic fibers; or (3) by bonding with a cementing medium such as a natural or synthetic resin.

As used herein, the term “liquid” refers to a substance having a fluid consistency. For example, and not limitation, liquids can include water, oils, solvents, bodily fluids such as urine or blood, wet foodstuff such as beverages and soups, disinfectants, lotions, and cleaning solutions.

As used herein, the term “weight percent” is meant to refer to either (i) the quantity by weight of a constituent/component in the material as a percentage of the weight of a layer of the material; or (ii) to the quantity by weight of a constituent/component in the material as a percentage of the weight of the final nonwoven material or product.

The term “basis weight” as used herein refers to the quantity by weight of a compound over a given area. Examples of the units of measure include grams per square meter as identified by the acronym “gsm”.

As used in the specification and the appended claims, the singular forms “a,” “an” and “the” include plural referents unless the context clearly dictates otherwise. Thus, for example, reference to “a compound” includes mixtures of compounds.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to systems or processes, the term can mean within an order of magnitude, preferably within 5-fold, and more preferably within 2-fold, of a value.

### Fibers

The nonwoven material of the presently disclosed subject matter comprises fibers. The fibers can be natural, synthetic, or a mixture thereof. In one embodiment, the fibers can be cellulose-based fibers, one or more synthetic fibers, or a mixture thereof.

### Cellulose Fibers

Any cellulose fibers known in the art, including cellulose fibers of any natural origin, such as those derived from wood pulp or regenerated cellulose, can be used in a cellulosic layer. In certain embodiment, cellulose fibers include, but are not limited to, digested fibers, such as kraft, prehydrolyzed kraft, soda, sulfite, chemi-thermal mechanical, and thermo-mechanical treated fibers, derived from softwood, hardwood or cotton linters. In other embodiments, cellulose fibers include, but are not limited to, kraft digested fibers, including prehydrolyzed kraft digested fibers. Non-limiting examples of cellulose fibers suitable for use in this subject matter are the cellulose fibers derived from softwoods, such as pines, firs, and spruces. Other suitable cellulose fibers include, but are not limited to, those derived from Esparto grass, bagasse, kemp, flax, hemp, kenaf, and other lignaceous and cellulosic fiber sources. Suitable cellulose fibers include, but are not limited to, bleached Kraft southern pine fibers sold under the trademark FOLEY FLUFFS® (Buckeye Technologies Inc., Memphis, Tenn.). Additionally, fibers sold under the trademark CELLU TISSUE® (e.g., Grade 3024) (Clearwater Paper Corporation, Spokane, Wash.) are utilized in certain aspects of the disclosed subject matter.

The nonwoven materials of the disclosed subject matter can also include, but are not limited to, a commercially available bright fluff pulp including, but not limited to, southern softwood fluff pulp (such as Treated FOLEY FLUFFS®) northern softwood sulfite pulp (such as T 730 from Weyerhaeuser), or hardwood pulp (such as eucalyptus). While certain pulps may be preferred based on a variety of factors, any absorbent fluff pulp or mixtures thereof can be used. In certain embodiments, wood cellulose, cotton linter pulp, chemically modified cellulose such as crosslinked cellulose fibers and highly purified cellulose fibers can be used. Non-limiting examples of additional pulps are FOLEY FLUFFS® FFTAS (also known as FFTAS or Buckeye Technologies FFT-AS pulp), and Weyco CF401.

Other suitable types of cellulose fiber include, but are not limited to, chemically modified cellulose fibers. In particular embodiments, the modified cellulose fibers are crosslinked cellulose fibers. U.S. Pat. Nos. 5,492,759; 5,601,921; 6,159,335, all of which are hereby incorporated by reference in their entireties, relate to chemically treated cellulose fibers useful in the practice of this disclosed subject matter. In certain embodiments, the modified cellulose fibers comprise a polyhydroxy compound. Non-limiting examples of polyhydroxy compounds include glycerol, trimethylolpropane, pentaerythritol, polyvinyl alcohol, partially hydrolyzed polyvinyl acetate, and fully hydrolyzed polyvinyl acetate. In certain embodiments, the fiber is treated with a polyvalent cation-containing compound. In one embodiment, the polyvalent cation-containing compound is present in an amount from about 0.1 weight percent to about 20 weight percent based on the dry weight of the untreated fiber. In particular embodiments, the polyvalent cation containing compound is a polyvalent metal ion salt. In certain embodiments, the polyvalent cation containing compound is selected from the group consisting of aluminum, iron, tin, salts thereof, and mixtures thereof. Any polyvalent metal salt including transition metal salts may be used. Non-limiting examples of suitable polyvalent metals include beryllium, magnesium, calcium, strontium, barium, titanium, zirconium, vanadium, chromium, molybdenum, tungsten, manganese, iron, cobalt, nickel, copper, zinc, aluminum and tin. Preferred ions include aluminum, iron and tin. The preferred metal ions have oxidation states of +3 or +4. Any salt containing the polyvalent metal ion may be employed. Non-limiting examples of suitable inorganic salts of the above metals include chlorides, nitrates, sulfates, borates, bromides, iodides, fluorides, nitrides, perchlorates, phosphates, hydroxides, sulfides, carbonates, bicarbonates, oxides, alkoxides phenoxides, phosphites, and hypophosphites. Non-limiting examples of suitable organic salts of the above metals include formates, acetates, butyrates, hexanoates, adipates, citrates, lactates, oxalates, propionates, salicylates, glycinates, tartrates, glycolates, sulfonates, phosphonates, glutamates, octanoates, benzoates, gluconates, maleates, succinates, and 4,5-dihydroxy-benzene-1,3-disulfonates. In addition to the polyvalent metal salts, other compounds such as complexes of the above salts include, but are not limited to, amines, ethylenediaminetetra-acetic acid (EDTA), diethylenetriaminepenta-acetic acid (DIPA), nitrilotri-acetic acid (NTA), 2,4-pentanedione, and ammonia may be used.

In one embodiment, the cellulose pulp fibers are chemically modified cellulose pulp fibers that have been softened or plasticized to be inherently more compressible than unmodified pulp fibers. The same pressure applied to a plasticized pulp web will result in higher density than when applied to an unmodified pulp web. Additionally, the densified web of plasticized cellulose fibers is inherently softer than a similar density web of unmodified fiber of the same wood type. Softwood pulps may be made more compressible using cationic surfactants as debonders to disrupt interfiber associations. Use of one or more debonders facilitates the disintegration of the pulp sheet into fluff in the airlaid process. Examples of debonders include, but are not limited to, those disclosed in U.S. Pat. Nos. 4,432,833, 4,425,186 and 5,776,308, all of which are hereby incorporated by reference in their entireties. One example of a debonder-treated cellulose pulp is FFLE+. Plasticizers for cellulose, which can be added to a pulp slurry prior to forming wetlaid sheets, can also be used to soften pulp, although they act by a different mechanism than debonding agents. Plasticizing agents act within the fiber, at the cellulose molecule, to make flexible or soften amorphous regions. The resulting fibers are characterized as limp. Since the plasticized fibers lack stiffness, the comminuted pulp is easier to densify compared to fibers not treated with plasticizers. Plasticizers include, but are not limited to, polyhydric alcohols such as glycerol, low molecular weight polyglycol such as polyethylene glycols, and polyhydroxy compounds. These and other plasticizers are described and exemplified in U.S. Pat. Nos. 4,098,996, 5,547,541 and 4,731,269, all of which are hereby incorporated by reference in their entireties. Ammonia, urea, and alkylamines are also known to plasticize wood products, which mainly contain cellulose (A. J. Stamm, Forest Products Journal 5(6):413, 1955, hereby incorporated by reference in its entirety).

In particular embodiments of the disclosed subject matter, the following cellulose is used: GP4723, a fully treated pulp (available from Georgia-Pacific); GP4725, a semi-treated pulp (available from Georgia-Pacific); Tencel (available from Lenzing); cellulose flax fibers; Danufil (available from Kelheim); Viloft (available from Kelheim); GP4865, an odor control semi-treated pulp (available from Georgia-Pacific); Grade 3024 Cellu Tissue (available from Clearwater); Brawny Industrial Flax 500 (available from Georgia-Pacific).

In certain embodiments, a particular layer can contain from about 5 gsm to about 150 gsm cellulose fibers, or from about 5 gsm to about 100 gsm cellulose fibers, or from about 10 gsm to about 50 gsm cellulose fibers. In particular embodiments, a layer can contain from about 7 gsm to about 40 gsm cellulose fibers, or from about 10 gsm to about 30 gsm cellulose fibers, or from about 15 gsm to about 24 gsm cellulose fibers.

### Synthetic Fibers

In addition to the use of cellulose fibers, the presently disclosed subject matter also contemplates the use of synthetic fibers. In one embodiment, the synthetic fibers comprise bicomponent and/or mono-component fibers. Bicomponent fibers having a core and sheath are known in the art. Many varieties are used in the manufacture of nonwoven materials, particularly those produced for use in airlaid techniques. Various bicomponent fibers suitable for use in the presently disclosed subject matter are disclosed in U.S. Pat. Nos. 5,372,885 and 5,456,982, both of which are hereby incorporated by reference in their entireties. Examples of bicomponent fiber manufacturers include, but are not limited to, Trevira (Bobingen, Germany), Fiber Innovation Technologies (Johnson City, Tenn.) and ES Fiber Visions (Athens, Ga.).

Bicomponent fibers can incorporate a variety of polymers as their core and sheath components. Bicomponent fibers that have a PE (polyethylene) or modified PE sheath typically have a PET (polyethylene terephthalate) or PP (polypropylene) core. In one embodiment, the bicomponent fiber has a core made of polyester and sheath made of polyethylene. In another embodiment, the bicomponent fiber has a core made of polypropylene and a sheath made of polyethylene.

The denier of the bicomponent fiber preferably ranges from about 1.0 dpf to about 4.0 dpf, and more preferably from about 1.5 dpf to about 2.5 dpf. The length of the bicomponent fiber can be from about 3 mm to about 36 mm, preferably from about 3 mm to about 12 mm, more preferably from about 3 mm to about 10. In particular embodiments, the length of the bicomponent fiber is from about 4 mm to about 8 mm, or about 6 mm. In a particular embodiment, the bicomponent fiber is Trevira T255 which contains a polyester core and a polyethylene sheath modified with maleic anhydride. T255 has been produced in a variety of deniers, cut lengths and core sheath configurations with preferred configurations having a denier from about 1.7 dpf to 2.0 dpf and a cut length of about 4 mm to 12 mm and a concentric core sheath configuration. In a specific embodiment, the bicomponent fiber is Trevira 1661, T255, 2.0 dpf and 6 mm in length. In an alternate embodiment, the bicomponent fiber is Trevira 1663, T255, 2.0 dpf and 3 mm in length.

Bicomponent fibers are typically fabricated commercially by melt spinning. In this procedure, each molten polymer is extruded through a die, for example, a spinneret, with subsequent pulling of the molten polymer to move it away from the face of the spinneret. This is followed by solidification of the polymer by heat transfer to a surrounding fluid medium, for example chilled air, and taking up of the now solid filament. Non-limiting examples of additional steps after melt spinning can also include hot or cold drawing, heat treating, crimping and cutting. This overall manufacturing process is generally carried out as a discontinuous two-step process that first involves spinning of the filaments and their collection into a tow that comprises numerous filaments. During the spinning step, when molten polymer is pulled away from the face of the spinneret, some drawing of the filament does occur which can also be called the draw-down. This is followed by a second step where the spun fibers are drawn or stretched to increase molecular alignment and crystallinity and to give enhanced strength and other physical properties to the individual filaments. Subsequent steps can include, but are not limited to, heat setting, crimping and cutting of the filament into fibers. The drawing or stretching step can involve drawing the core of the bicomponent fiber, the sheath of the bicomponent fiber or both the core and the sheath of the bicomponent fiber depending on the materials from which the core and sheath are comprised as well as the conditions employed during the drawing or stretching process.

Bicomponent fibers can also be formed in a continuous process where the spinning and drawing are done in a continuous process. During the fiber manufacturing process it is desirable to add various materials to the fiber after the melt spinning step at various subsequent steps in the process. These materials can be referred to as “finish” and be comprised of active agents such as, but not limited to, lubricants and anti-static agents. The finish is typically delivered via an aqueous based solution or emulsion. Finishes can provide desirable properties for both the manufacturing of the bicomponent fiber and for the user of the fiber, for example in an airlaid or wetlaid process.

Numerous other processes are involved before, during and after the spinning and drawing steps and are disclosed in U.S. Pat. Nos. 4,950,541, 5,082,899, 5,126,199, 5,372,885, 5,456,982, 5,705,565, 2,861,319, 2,931,091, 2,989,798, 3,038,235, 3,081,490, 3,117,362, 3,121,254, 3,188,689, 3,237,245, 3,249,669, 3,457,342, 3,466,703, 3,469,279, 3,500,498, 3,585,685, 3,163,170, 3,692,423, 3,716,317, 3,778,208, 3,787,162, 3,814,561, 3,963,406, 3,992,499, 4,052,146, 4,251,200, 4,350,006, 4,370,114, 4,406,850, 4,445,833, 4,717,325, 4,743,189, 5,162,074, 5,256,050, 5,505,889, 5,582,913, and 6,670,035, all of which are hereby incorporated by reference in their entireties.

The presently disclosed subject matter can also include, but are not limited to, articles that contain bicomponent fibers that are partially drawn with varying degrees of draw or stretch, highly drawn bicomponent fibers and mixtures thereof. These can include, but are not limited to, a highly drawn polyester core bicomponent fiber with a variety of sheath materials, specifically including a polyethylene sheath such as Trevira T255 (Bobingen, Germany) or a highly drawn polypropylene core bicomponent fiber with a variety of sheath materials, specifically including a polyethylene sheath such as ES FiberVisions AL-Adhesion-C (Varde, Denmark). Additionally, Trevira T265 bicomponent fiber (Bobingen, Germany), having a partially drawn core with a core made of polybutylene terephthalate (PBT) and a sheath made of polyethylene can be used. The use of both partially drawn and highly drawn bicomponent fibers in the same structure can be leveraged to meet specific physical and performance properties based on how they are incorporated into the structure.

The bicomponent fibers of the presently disclosed subject matter are not limited in scope to any specific polymers for either the core or the sheath as any partially drawn core bicomponent fiber can provide enhanced performance regarding elongation and strength. The degree to which the partially drawn bicomponent fibers are drawn is not limited in scope as different degrees of drawing will yield different enhancements in performance. The scope of the partially drawn bicomponent fibers encompasses fibers with various core sheath configurations including, but not limited to concentric, eccentric, side by side, islands in a sea, pie segments and other variations. The relative weight percentages of the core and sheath components of the total fiber can be varied. In addition, the scope of this subject matter covers the use of partially drawn homopolymers such as polyester, polypropylene, nylon, and other melt spinnable polymers. The scope of this subject matter also covers multicomponent fibers that can have more than two polymers as part of the fiber structure.

In particular embodiments, the bicomponent fibers in a particular layer comprise from about 50 to about 100 percent by weight of the layer. The bicomponent layer can contain from about 1 gsm to about 30 gsm bicomponent fibers, or about 1 gsm to about 20 gsm bicomponent fibers, or from about 2 gsm to about 10 gsm bicomponent fibers, or about 2 gsm to about 8 gsm bicomponent fibers. In certain embodiments, the bicomponent layer contains from about 4 gsm to about 20 gsm bicomponent fibers. In alternative embodiments, the bicomponent layer contains from about 10 gsm to about 50 gsm bicomponent fibers, or from about 12 gsm to about 40 gsm bicomponent fibers, or from about 20 gsm to about 30 gsm bicomponent fibers.

In particular embodiments, the bicomponent fibers are low dtex staple bicomponent fibers in the range of about 0.5 dtex to about 20 dtex. In certain embodiments, the dtex value can range from about 1.3 dtex to about 15 dtex, or from about 1.5 dtex to about 10 dtex, or from about 1.7 dtex to about 6.7 dtex, or from about 2.2 dtex to about 5.7 dtex. In certain embodiments, the dtex value is 1.3 dtex, 2.2 dtex, 3.3 dtex, 5.7 dtex, 6.7 dtex, or 10 dtex.

Other synthetic fibers suitable for use in various embodiments as fibers or as bicomponent binder fibers include, but are not limited to, fibers made from various polymers including, by way of example and not by limitation, acrylic, polyamides (including, but not limited to, Nylon 6, Nylon 6/6, Nylon 12, polyaspartic acid, polyglutamic acid), polyamines, polyimides, polyacrylics (including, but not limited to, polyacrylamide, polyacrylonitrile, esters of methacrylic acid and acrylic acid), polycarbonates (including, but not limited to, polybisphenol A carbonate, polypropylene carbonate), polydienes (including, but not limited to, polybutadiene, polyisoprene, polynorbomene), polyepoxides, polyesters (including, but not limited to, polyethylene terephthalate, polybutylene terephthalate, polytrimethylene terephthalate, polycaprolactone, polyglycolide, polylactide, polyhydroxybutyrate, polyhydroxyvalerate, polyethylene adipate, polybutylene adipate, polypropylene succinate), polyethers (including, but not limited to, polyethylene glycol (polyethylene oxide), polybutylene glycol, polypropylene oxide, polyoxymethylene (paraformaldehyde), polytetramethylene ether (polytetrahydrofuran), polyepichlorohydrin), polyfluorocarbons, formaldehyde polymers (including, but not limited to, urea-formaldehyde, melamine-formaldehyde, phenol formaldehyde), natural polymers (including, but not limited to, cellulosics, chitosans, lignins, waxes), polyolefins (including, but not limited to, polyethylene, polypropylene, polybutylene, polybutene, polyoctene), polyphenylenes (including, but not limited to, polyphenylene oxide, polyphenylene sulfide, polyphenylene ether sulfone), silicon containing polymers (including, but not limited to, polydimethyl siloxane, polycarbomethyl silane), polyurethanes, polyvinyls (including, but not limited to, polyvinyl butyral, polyvinyl alcohol, esters and ethers of polyvinyl alcohol, polyvinyl acetate, polystyrene, polymethylstyrene, polyvinyl chloride, polyvinyl pryrrolidone, polymethyl vinyl ether, polyethyl vinyl ether, polyvinyl methyl ketone), polyacetals, polyarylates, and copolymers (including, but not limited to, polyethylene-co-vinyl acetate, polyethylene-co-acrylic acid, polybutylene terephthalate-co-polyethylene terephthalate, polylauryllactam-block-polytetrahydrofuran), polybutylene succinate and polylactic acid based polymers.

In specific embodiments, the synthetic fiber layer contains a high dtex staple fibers in the range of about 2 to about 20 dtex. In certain embodiments, the dtex value can range from about 2 dtex to about 15 dtex, or from about 2 dtex to about 10 dtex. In particular embodiments, the fiber can have a dtex value of about 6.7 dtex.

In other specific embodiments, the synthetic layer contains synthetic filaments. The synthetic filaments can be formed by spinning and/or extrusion processes. For example, such processes can be similar to the methods described above with reference to melt spinning processes. The synthetic filaments can include one or more continuous strands. In certain embodiments, the synthetic filaments can include polypropylene.

In particular embodiments, polyester (PET) fibers such as Trevira Type 245, are used in a synthetic fiber layer comprising from about 50 to about 100 percent by weight of the layer. The synthetic fiber layer contains from about 5 gsm to about 50 gsm synthetic fibers, or from about 10 gsm to about 20 gsm synthetic fibers, or from about 12 to about 16 synthetic fibers, or from about 13 gsm to about 15 gsm synthetic fibers.

### Binders

Suitable binders include, but are not limited to, liquid binders and powder binders. Non-limiting examples of liquid binders include emulsions, solutions, or suspensions of binders. Non-limiting examples of binders include polyethylene powders, copolymer binders, vinylacetate ethylene binders, styrene-butadiene binders, urethanes, urethane-based binders, acrylic binders, thermoplastic binders, natural polymer based binders, and mixtures thereof.

Suitable binders include, but are not limited to, copolymers, vinylacetate ethylene (“VAE”) copolymers, which can have a stabilizer such as Wacker Vinnapas 192, Wacker Vinnapas EF 539, Wacker Vinnapas EP907, Wacker Vinnapas EP129, Celanese Duroset E130, Celanese Dur-O-Set Elite 130 25-1813 and Celanese Dur-O-Set TX-849, Celanese 75-524A, polyvinyl alcohol-polyvinyl acetate blends such as Wacker Vinac 911, vinyl acetate homopolyers, polyvinyl amines such as BASF Luredur, acrylics, cationic acrylamides, polyacryliamides such as Bercon Berstrength 5040 and Bercon Berstrength 5150, hydroxyethyl cellulose, starch such as National Starch CATO RTM 232, National Starch CATO RTM 255, National Starch Optibond, National Starch Optipro, or National Starch OptiPLUS, guar gum, styrene-butadienes, urethanes, urethane-based binders, thermoplastic binders, acrylic binders, and carboxymethyl cellulose such as Hercules Aqualon CMC. In certain embodiments, the binder is a natural polymer based binder. Non-limiting examples of natural polymer based binders include polymers derived from starch, cellulose, chitin, and other polysaccharides.

In certain embodiments, the binder is water-soluble. In one embodiment, the binder is a vinylacetate ethylene copolymer. One non-limiting example of such copolymers is EP907 (Wacker Chemicals, Munich, Germany). Vinnapas EP907 can be applied at a level of about 10% solids incorporating about 0.75% by weight Aerosol OT (Cytec Industries, West Paterson, N.J.), which is an anionic surfactant. Other classes of liquid binders such as styrene-butadiene and acrylic binders can also be used.

In certain embodiments, the binder is not water-soluble. Examples of these binders include, but are not limited to, Vinnapas 124 and 192 (Wacker), which can have an opacifier and whitener, including, but not limited to, titanium dioxide, dispersed in the emulsion. Other binders include, but are not limited to, Celanese Emulsions (Bridgewater, N.J.) Elite 22 and Elite 33.

In certain embodiments, the binder is a thermoplastic binder. Such thermoplastic binders include, but are not limited to, any thermoplastic polymer which can be melted at temperatures which will not extensively damage the cellulose fibers. Preferably, the melting point of the thermoplastic binding material will be less than about 175° C. Examples of suitable thermoplastic materials include, but are not limited to, suspensions of thermoplastic binders and thermoplastic powders. In particular embodiments, the thermoplastic binding material can be, for example, polyethylene, polypropylene, polyvinylchloride, and/or polyvinylidene chloride.

In particular embodiments, the vinylacetate ethylene binder is non-crosslinkable. In one embodiment, the vinylacetate ethylene binder is crosslinkable. In certain embodiments, the binder is WD4047 urethane-based binder solution supplied by HB Fuller. In one embodiment, the binder is Michem Prime 4983-45N dispersion of ethylene acrylic acid (“EAA”) copolymer supplied by Michelman. In certain embodiments, the binder is Dur-O-Set Elite 22LV emulsion of VAE binder supplied by Celanese Emulsions (Bridgewater, N.J.). As noted above, in particular embodiments, the binder is crosslinkable. It is also understood that crosslinkable binders are also known as permanent wet strength binders. A permanent wet-strength binder includes, but is not limited to, Kymene® (Hercules Inc., Wilmington, Del.), Parez® (American Cyanamid Company, Wayne, N.J.), Wacker Vinnapas or AF192 (Wacker Chemie AG, Munich, Germany), or the like. Various permanent wet-strength agents are described in U.S. Pat. No. 2,345,543, U.S. Pat. No. 2,926,116, and U.S. Pat. No. 2,926,154, the disclosures of which are incorporated by reference in their entirety. Other permanent wet-strength binders include, but are not limited to, polyamine-epichlorohydrin, polyamide epichlorohydrin or polyamide-amine epichlorohydrin resins, which are collectively termed “PAE resins”. Non-limiting exemplary permanent wet-strength binders include Kymene 557H or Kymene 557LX (Hercules Inc., Wilmington, Del.) and have been described in U.S. Pat. No. 3,700,623 and U.S. Pat. No. 3,772,076, which are incorporated herein in their entirety by reference thereto.

Alternatively, in certain embodiments, the binder is a temporary wet-strength binder. The temporary wet-strength binders include, but are not limited to, Hercobond® (Hercules Inc., Wilmington, Del.), Parez® 750 (American Cyanamid Company, Wayne, N.J.), Parez® 745 (American Cyanamid Company, Wayne, N.J.), or the like. Other suitable temporary wet-strength binders include, but are not limited to, dialdehyde starch, polyethylene imine, mannogalactan gum, glyoxal, and dialdehyde mannogalactan. Other suitable temporary wet-strength agents are described in U.S. Pat. No. 3,556,932, U.S. Pat. No. 5,466,337, U.S. Pat. No. 3,556,933, U.S. Pat. No. 4,605,702, U.S. Pat. No. 4,603,176, U.S. Pat. No. 5,935,383, and U.S. Pat. No. 6,017,417, all of which are incorporated herein in their entirety by reference thereto.

In certain embodiments, binders are applied as emulsions in amounts ranging from about 1 gsm to about 4 gsm, or from about 1.3 gsm to about 2.8 gsm, or from about 2 gsm to about 3 gsm. Binder can be applied to one side of a fibrous layer, preferably an externally facing layer. Alternatively, binder can be applied to both sides of a layer, in equal or disproportionate amounts.

### Other Additives

The materials of the presently disclosed subject matter can also contain other additives. For example, the materials can contain superabsorbent polymer (SAP). The types of superabsorbent polymers which may be used in the disclosed subject matter include, but are not limited to, SAPs in their particulate form such as powder, irregular granules, spherical particles, staple fibers and other elongated particles. U.S. Pat. Nos. 5,147,343; 5,378,528; 5,795,439; 5,807,916; 5,849,211, and 6,403,857, which are hereby incorporated by reference in their entireties, describe various superabsorbent polymers and methods of making superabsorbent polymers. One example of a superabsorbent polymer forming system is crosslinked acrylic copolymers of metal salts of acrylic acid and acrylamide or other monomers such as 2-acrylamido-2-methylpropanesulfonic acid. Many conventional granular superabsorbent polymers are based on poly(acrylic acid) which has been crosslinked during polymerization with any of a number of multi-functional co-monomer crosslinking agents well-known in the art. Examples of multi-functional crosslinking agents are set forth in U.S. Pat. Nos. 2,929,154; 3,224,986; 3,332,909; 4,076,673, which are incorporated herein by reference in their entireties. For instance, crosslinked carboxylated polyelectrolytes can be used to form superabsorbent polymers. Other water-soluble polyelectrolyte polymers are known to be useful for the preparation of superabsorbents by crosslinking, these polymers include: carboxymethyl starch, carboxymethyl cellulose, chitosan salts, gelatine salts, etc. They are not, however, commonly used on a commercial scale to enhance absorbency of dispensable absorbent articles mainly due to their higher cost. Superabsorbent polymer granules useful in the practice of this subject matter are commercially available from a number of manufacturers, such as BASF, Dow Chemical (Midland, Mich.), Stockhausen (Greensboro, N.C.), Chemdal (Arlington Heights, Ill.), and Evonik (Essen, Germany). Non-limiting examples of SAP include a surface crosslinked acrylic acid based powder such as Stockhausen 9350 or SX70, BASF HySorb FEM 33N, or Evonik Favor SXM 7900.

In certain embodiments, SAP can be used in a layer in amounts ranging from about 5% to about 50% based on the total weight of the structure. In certain embodiments, the amount of SAP in a layer can range from about 10 gsm to about 50 gsm, or from about 12 gsm to about 40 gsm, or from about 15 gsm to about 25 gsm.

### Nonwoven Materials

The presently disclosed subject matter provides for improved nonwoven materials with many advantages over various commercially available materials. The presently disclosed materials have a significantly reduced absorbent mass, with an ability to achieve comparable or improved overall absorbency performance. The absorbency performance is measured by better fluid acquisition or improved dryness characteristics, while maintaining similar basis weights to commercially available products.

The presently disclosed subject matter provides for a nonwoven material. In certain embodiments, the nonwoven material includes at least two layers, at least three layers, at least four layers, at least five layers, or at least six layers.

In certain embodiments, the nonwoven material is a nonwoven acquisition material that comprises at least two layers, wherein each layer comprises a specific fibrous content.

In specific embodiments, the nonwoven acquisition material can be a two-layer nonwoven structure. The nonwoven acquisition material can contain a synthetic fiber layer and a cellulosic fiber layer. In certain embodiments, the synthetic fiber layer is a bicomponent fiber layer. In other embodiments, the nonwoven acquisition material contains two synthetic fiber layers. In specific embodiments, one or more synthetic fiber layers contains synthetic filaments.

In a particular embodiment, the nonwoven acquisition material can be a two-layer nonwoven structure having a synthetic fiber layer and a cellulosic fiber layer. The first layer can contain from about 10 gsm to about 50 gsm of synthetic fibers. The synthetic fibers can be polyethylene terephthalate (PET) fibers. The first layer can be bonded on at least a portion of its outer surface with binder. In alternative embodiments, the first layer can contain from about 10 gsm to about 50 gsm of bicomponent fibers having an eccentric core sheath configuration. The second layer can contain from about 10 gsm to about 100 gsm of cellulose fibers. The second layer can be bonded on at least a portion of its outer surface with binder.

In another particular embodiment, the nonwoven acquisition material can be a two-layer nonwoven structure having two synthetic fiber layers. The first layer can contain from about 10 gsm to about 50 gsm of synthetic fibers. The synthetic fibers can be polyethylene terephthalate (PET) fibers. The first layer can be bonded on at least a portion of its outer surface with binder. In alternative embodiments, the first layer can contain from about 10 gsm to about 50 gsm of bicomponent fibers having an eccentric core sheath configuration. The second layer can contain synthetic filaments.

In alternative embodiments, the nonwoven acquisition material comprises at least three layers, wherein each layer comprises a specific fibrous content. In specific embodiments, the nonwoven acquisition material contains a cellulosic fiber layer, a bicomponent fiber layer, and a synthetic fiber layer. In certain embodiments, the layers are bonded on at least a portion of at least one of their outer surfaces with binder. It is not necessary that the binder chemically bond with a portion of the layer, although it is preferred that the binder remain associated in close proximity with the layer, by coating, adhering, precipitation, or any other mechanism such that it is not dislodged from the layer during normal handling of the layer. For convenience, the association between the layer and the binder discussed above can be referred to as the bond, and the compound can be said to be bonded to the layer.

In one embodiment, the first layer comprises synthetic fibers. In certain embodiments, the first layer is coated with binder on its outer surface. In other certain embodiments, the first layer comprises bicomponent fibers. A second layer disposed adjacent to the first layer, comprises bicomponent fibers. A third layer disposed adjacent to the second layer comprises cellulose fibers. In an alternate embodiment, the third layer contains synthetic fibers. In a particular embodiment, third layer is coated with binder on its outer surface.

In another embodiment, the first layer contains from about 5 gsm to about 50 gsm, or from about 10 gsm to about 20 gsm of synthetic fibers. Where the synthetic fibers are bicomponent fibers, the first layer can contain from about 10 to about 50 gsm, or from about 12 gsm to about 40 gsm, or from about 20 gsm to about 30 gsm bicomponent fibers. In certain embodiments, the second layer contains from about 1 gsm to about 50 gsm, or from about 4 gsm to about 40 gsm, or from about 12 gsm to about 20 gsm of bicomponent fibers. In certain embodiments, the third layer contains from about 5 gsm to about 100 gsm, or from about 10 gsm to about 50 gsm of cellulose fibers, or in the alternative synthetic fibers.

In a particular embodiment, the nonwoven acquisition material can be a three-layer nonwoven structure having a first synthetic fiber layer, a second synthetic fiber layer, and a third cellulosic fiber layer. The first layer can contain from about 10 gsm to about 50 gsm of synthetic fibers. The synthetic fibers can be polyethylene terephthalate (PET) fibers. The first layer can be bonded on at least a portion of its outer surface with binder. In alternative embodiments, the first layer can contain from about 10 gsm to about 50 gsm of bicomponent fibers having an eccentric core sheath configuration. The second layer can contain from about 4 gsm to about 20 gsm of bicomponent fibers. The third layer can contain from about 10 gsm to about 100 gsm of cellulose fibers. The third layer can be bonded on at least a portion of its outer surface with binder.

In another particular embodiment, the nonwoven acquisition material can be a two-layer nonwoven structure having a first synthetic fiber layer, a second synthetic fiber layer, and a third synthetic fiber layer. The first layer can contain from about 10 gsm to about 50 gsm of synthetic fibers. The synthetic fibers can be polyethylene terephthalate (PET) fibers. The first layer can be bonded on at least a portion of its outer surface with binder. In alternative embodiments, the first layer can contain from about 10 gsm to about 50 gsm of bicomponent fibers having an eccentric core sheath configuration. The second layer can contain from about 4 gsm to about 20 gsm of bicomponent fibers. The third layer can contain synthetic filaments.

In another embodiment of the presently disclosed subject matter, the nonwoven acquisition layer has at least four layers, wherein each layer has a specific fibrous content. In certain embodiments, the first layer contains synthetic fiber. In certain embodiments, the first layer is coated with binder on its outer surface. A second layer disposed adjacent to the first layer, contains bicomponent fibers. A third layer disposed adjacent to the second layer contains cellulose fibers and bicomponent fibers. A fourth layer disposed adjacent to the third layer contains cellulose fibers coated with binder on its outer surface.

In a specific embodiment, the first layer comprises from about 5 gsm to about 50 gsm, or from about 10 gsm to about 20 gsm of synthetic fibers. In certain embodiments, the second layer comprises from about 1 gsm to about 20 gsm, or from about 2 gsm to about 10 gsm of bicomponent fibers. In certain embodiments, the third layer comprises from about 7 gsm to about 40 gsm, or from about 10 gsm to about 30 gsm, or from about 15 gsm to about 24 gsm of cellulose fibers and from about 1 gsm to about 20 gsm of bicomponent fibers. In certain embodiments, the fourth layer comprises from about from about 5 gsm to about 100 gsm, or from about 10 gsm to about 50 gsm of cellulose fibers.

### Absorbent Cores

In another aspect, the presently disclosed subject matter provides for a multi-layer nonwoven material containing at least one layer adjacent to an absorbent core. In certain embodiments, the absorbent core has at least five layers, wherein each layer has a specific fibrous content. In certain embodiments, the first layer contains cellulose fibers, the second layer contains SAP, the third layer contains cellulose fibers, the fourth layer contains SAP, and the fifth layer contains cellulose fibers. In certain embodiments, one or more of the first layer, third layer, and/or fifth layer can further include bicomponent fibers. In certain embodiments, the nonwoven material can further include at least one additional layer adjacent to the absorbent core. In particular embodiments, the additional layer contains synthetic fibers.

In a specific embodiment, the first layer of the absorbent core contains from about 5 gsm to about 100 gsm, or from about 10 gsm to about 50 gsm of cellulose fibers. In certain embodiments, the second layer contains from about 10 gsm to about 50 gsm, or from about 12 gsm to about 40 gsm, or from about 15 gsm to about 25 gsm of SAP particles. In certain embodiments, the third layer contains from about 5 gsm to about 100 gsm, or from about 10 gsm to about 50 gsm cellulose fibers. In certain embodiments, the fourth layer contains from about 10 gsm to about 50 gsm, or from about 12 gsm to about 40 gsm, or from about 15 gsm to about 25 gsm of SAP particles. In certain embodiments, the fifth layer contains from about 5 gsm to about 100 gsm, or from about 10 gsm to about 50 gsm cellulose fibers. In certain embodiments, the cellulose fibers can be cellulose pulp. For example and not limitation, the cellulose fibers can be a hardwood pulp, such as eucalyptus pulp.

In certain embodiments, the nonwoven material includes at least one additional layer adjacent to the absorbent core. In certain embodiments, an additional layer contains from about 5 gsm to about 50 gsm, or from about 10 gsm to about 20 gsm of synthetic fibers. In particular embodiments, the synthetic fibers can be polyethylene terephthalate (PET) fibers. The additional layer can be bonded on at least a portion of its outer surface with binder. In alternative embodiments, the additional layer can contain from about 10 gsm to about 50 gsm of bicomponent fibers having an eccentric core sheath configuration.

### Features of the Nonwoven Materials

In certain embodiments of the presently disclosed subject matter, at least a portion of at least one outer layer is coated with binder. In particular embodiments of the disclosed subject matter, at least a portion of each outer layer is coated with binder. In particular embodiments, the first and third layers are coated with a binder in amounts ranging from about 1 gsm to about 4 gsm, or from about 1.3 gsm to about 2.8 gsm, or from about 2 gsm to about 3 gsm.

In certain embodiments of the nonwoven material, the range of basis weight of the overall structure is from about 5 gsm to about 600 gsm, or from about 5 gsm to about 400 gsm, or from about 10 gsm to about 400 gsm, or from about 20 gsm to 300 gsm, or from about 10 gsm to about 200 gsm, or from about 20 gsm to about 200 gsm, or from about 30 gsm to about 200 gsm, or from about 40 gsm to about 200 gsm. In certain embodiments where an absorbent core is present, the range of basis weight of the overall structure can be from about 10 gsm to about 1000 gsm, or from about 50 gsm to about 800 gsm, or from about 100 gsm to about 600 gsm.

The caliper of the nonwoven material refers to the caliper of the entire nonwoven material, inclusive of all layers. In certain embodiments, the caliper of the material ranges from about 0.5 mm to about 8.0 mm, or from about 0.5 mm to about 4 mm, or from about 0.5 mm to about 3.0 mm, or from about 0.5 mm to about 2.0 mm, or from about 0.7 mm to about 1.5 mm.

The presently disclosed nonwoven materials can have improved mechanical properties. For example, the nonwoven materials can have a tensile strength at peak load of greater than about 400 grams-force per inch (G/in), greater than about 500 G/in, greater than about 540 G/in, greater than about 570 G/in, greater than about 600 G/in, greater than about 630 G/in, greater than about 650 G/in, greater than about 670 G/in, or greater than about 690 G/in. Additionally, the nonwoven materials can have a percent elongation at peak load of greater than about 15%, greater than about 18%, greater than about 20%, greater than about 22%, greater than about 24%, greater than about 26%, greater than about 28%, or greater than about 30%.

The presently disclosed nonwoven materials can have improved fluid acquisition characteristics. For example, the nonwoven materials can absorb a fluid with minimal runoff. In certain embodiments, runoff from the nonwoven materials will be less than about 40%, less than about 30%, less than about 20%, or less than about 10% of the original amount of fluid applied to the nonwoven material. A person having ordinary skill in the art will appreciate that the amount of runoff, as well as any other absorbency characteristics of a nonwoven material, can vary. For example, the observed absorbency characteristics can vary based on the amount of fluid and the surface area of the nonwoven material. Additionally, when the nonwoven materials contain an absorbent core, the materials can have improved fluid acquisition characteristics. Furthermore, the nonwoven materials of the presently disclosed subject matter can quickly absorb a fluid. In certain embodiments, a nonwoven material as described above can absorb a fluid in less than about 60 seconds, less than about 45 seconds, or less than about 30 seconds. In particular embodiments, the nonwoven materials can absorb a fluid in less than about 26 seconds. The time it takes for a material to absorb a fluid can be called an “acquisition time.” For example, and not limitation, the acquisition time can be measured using the procedures described in Examples 3, 11, and 14 below.

Furthermore, the presently disclosed nonwoven materials can have improved dryness characteristics, indicating improved fluid retention. For example, after absorbing a fluid, the nonwoven materials can be pressed to measure the amount of fluid released. In certain embodiments, a rewet test or a humidity sensation test can be used to press the nonwoven material and measure the released fluid, as described in various examples below. In certain embodiments, less than about 3 g, less than about 2.8 g, or less than about 2.6 g is released. In other certain embodiments, less than about 1.8 g, less than about 1.6 g, or less than about 1.4 g is released. When the nonwoven materials contain an absorbent core, the materials can have increased fluid retention. In certain embodiments, less than about 500 mg, less than about 450 mg, less than about 400 mg, less than about 300 mg, less than about 200 mg, or less than about 150 mg is released from a nonwoven material having an absorbent core.

### Methods of Making the Materials

A variety of processes can be used to assemble the materials used in the practice of this disclosed subject matter to produce the materials, including but not limited to, traditional dry forming processes such as airlaying and carding or other forming technologies such as spunlace or airlace. Preferably, the materials can be prepared by airlaid processes. Airlaid processes include, but are not limited to, the use of one or more forming heads to deposit raw materials of differing compositions in selected order in the manufacturing process to produce a product with distinct strata. This allows great versatility in the variety of products which can be produced.

In one embodiment, the material is prepared as a continuous airlaid web. The airlaid web is typically prepared by disintegrating or defiberizing a cellulose pulp sheet or sheets, typically by hammermill, to provide individualized fibers. Rather than a pulp sheet of virgin fiber, the hammermills or other disintegrators can be fed with recycled airlaid edge trimmings and off-specification transitional material produced during grade changes and other airlaid production waste. Being able to thereby recycle production waste would contribute to improved economics for the overall process. The individualized fibers from whichever source, virgin or recycled, are then air conveyed to forming heads on the airlaid web-forming machine. A number of manufacturers make airlaid web forming machines suitable for use in the disclosed subject matter, including Dan-Web Forming of Aarhus, Denmark, M&J Fibretech A/S of Horsens, Denmark, Rando Machine Corporation, Macedon, N.Y. which is described in U.S. Pat. No. 3,972,092, Margasa Textile Machinery of Cerdanyola del Valles, Spain, and DOA International of Wels, Austria. While these many forming machines differ in how the fiber is opened and air-conveyed to the forming wire, they all are capable of producing the webs of the presently disclosed subject matter. The Dan-Web forming heads include rotating or agitated perforated drums, which serve to maintain fiber separation until the fibers are pulled by vacuum onto a foraminous forming conveyor or forming wire. In the M&J machine, the forming head is basically a rotary agitator above a screen. The rotary agitator may comprise a series or cluster of rotating propellers or fan blades. Other fibers, such as a synthetic thermoplastic fiber, are opened, weighed, and mixed in a fiber dosing system such as a textile feeder supplied by Laroche S. A. of Cours-La Ville, France. From the textile feeder, the fibers are air conveyed to the forming heads of the airlaid machine where they are further mixed with the comminuted cellulose pulp fibers from the hammer mills and deposited on the continuously moving forming wire. Where defined layers are desired, separate forming heads may be used for each type of fiber. Alternatively or additionally, one or more layers can be prefabricated prior to being combined with additional layers, if any.

The airlaid web is transferred from the forming wire to a calendar or other densification stage to densify the web, if necessary, to increase its strength and control web thickness. In one embodiment, the fibers of the web are then bonded by passage through an oven set to a temperature high enough to fuse the included thermoplastic or other binder materials. In a further embodiment, secondary binding from the drying or curing of a latex spray or foam application occurs in the same oven. The oven can be a conventional through-air oven, be operated as a convection oven, or may achieve the necessary heating by infrared or even microwave irradiation. In particular embodiments, the airlaid web can be treated with additional additives before or after heat curing.

### Applications and End Uses

The nonwoven materials of the disclosed subject matter can be used for any application known in the art. For example, the nonwoven materials can be used either alone or as a component in a variety of absorbent articles. In certain aspects, the nonwoven materials can be used in absorbent articles that absorb and retain body fluids. Such absorbent articles include baby diapers, adult incontinence products, sanitary napkins and the like.

In other aspects, the nonwoven materials can be used alone or as a component in other consumer products. For example, the nonwoven materials can be used in absorbent cleaning products, such wipes, sheets, towels and the like. By way of example, the nonwoven materials can be used as a disposable wipe for cleaning applications, including household, personal, and industrial cleaning applications. The absorbency of the nonwoven materials can aid in dirt and mess removal in such cleaning applications.

### EXAMPLES

The following examples are merely illustrative of the presently disclosed subject matter and they should not be considered as limiting the scope of the subject matter in any way.

### Example 1

### Three-Layer Nonwoven Acquisition Material

The present Example provides for a three-layer nonwoven acquisition material in accordance with the disclosed subject matter.

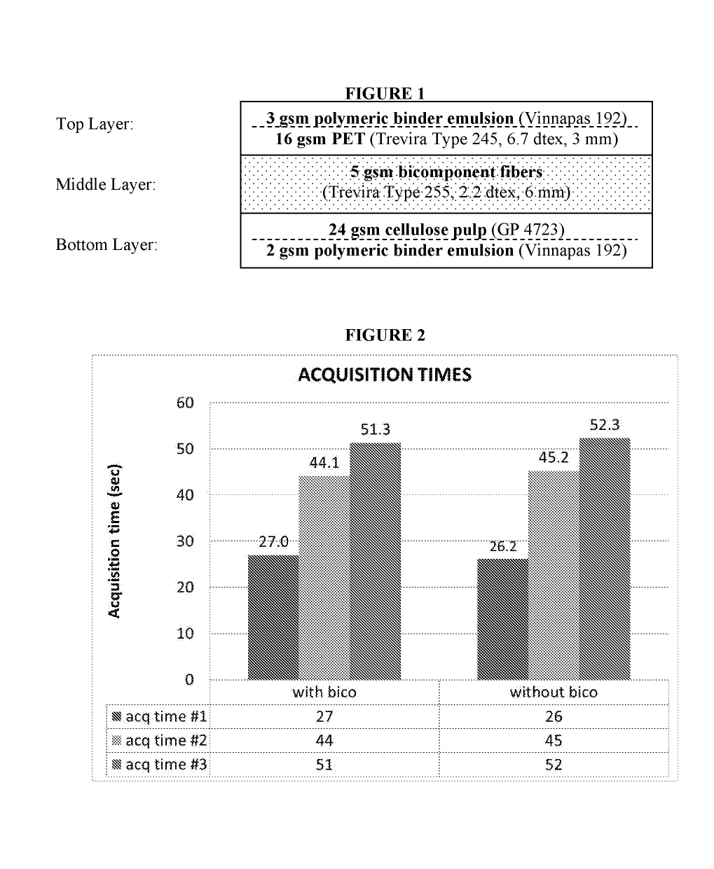

A first material was formed using a pilot drum-forming machine. The top layer of the three-layer, nonwoven acquisition material was composed of 16 gsm of PET fibers (Trevira Type 245, 6.7 dtex, 3 mm), which were bonded with a 3 gsm polymeric binder in the form of an emulsion (Vinnapas 192, Wacker). The middle layer was composed of 5 gsm bicomponent fibers (Trevira 1661, Type 255, 2.2 dtex, 6 mm). The bottom layer was composed of 24 gsm of cellulose (GP 4723, fully treated pulp from Georgia-Pacific), which was bonded with a 2 gsm polymeric binder in the form of an emulsion (Vinnapas 192, Wacker). The average thickness of the prepared structure was 0.76 mm. FIG. 1 gives a pictorial description of the first material composition. Three samples of the same material were prepared.

A second material was created having the same structure as the above structure, but without a bicomponent fiber layer underneath the PET fiber layer. The different basis weight of the cellulose bottom layer in this sample was 29 gsm. The average thickness of this structure was 0.68 mm. Again, three samples of the same material were prepared.

The tensile strength and elongation values of the acquisition material with and without bicomponent fiber were measured and recorded with the EJA Vantage Materials Tester (Thwing Albert Instrument Company) and the corresponding MAP4 software. Table 1 summarizes the data collected on the materials as an average of the three samples per material. Specifically, the Table shows the tensile strength at peak load and the elongation percentage (%) at peak load as an average of the three samples.

TABLE 1

| | Basis | Peak | % Elonga- |
|---|---|---|---|
| | Weight | Load | tion at |
| Description | (gsm) | (G/in) | Peak Load |
| Material 1 (with 5 gsm bicomponent | 50 | 650 | 26.7 |
| fiber Trevira 1661, Type 225) | | | |
| Material 2 (with no bicomponent | 50 | 541 | 18.3 |
| fiber) | | | |

The tensile strength of the first material (i.e., the structure with a bicomponent fiber layer) was higher than the tensile strength of the second material (i.e., the structure without the bicomponent fiber in the middle layer). High tensile strength can be desirable to increase product stability during the converting process.

Each of the acquisition layers from Table 1 was placed on top of a commercially available nonwoven core material (175 MBS3A, GP Steinfurt) to form a feminine hygiene composite. The composite was compressed with an 8.190 kg plate for 1 minute. The prepared composites were tested for their liquid acquisition performance using a prepared synthetic blood solution.

Synthetic blood was purchased from Johnson, Moen & Co. Inc. (Rochester, Minn.) (Lot #201141; February 2014). The synthetic blood had a surface tension of 40-44 dynes/cm (ASTM F23.40-F1670) and included various chemicals including ammonium polyacrylate polymer, Azo Red Dye, HPLC distilled water, among other proprietary ingredients. The synthetic blood was diluted with deionized water to a composition of 35% blood and 65% water.

Each feminine hygiene composite was insulted with 4 mL of the synthetic blood at a rate of 10 mL/min using a small pump three separate times. The three acquisition times were measured. The interval time between the insults was 10 minutes.

FIG. 2 illustrates the acquisition times of the two materials, with and without a bicomponent (“bico”) layer, for each of the three insults. The acquisition times of both materials were comparable.

Further, the rewet characteristics of each material were analyzed after measuring the three acquisition times. Three pieces of gauze (Covidien's Curity, all-purpose sponges, non-woven, 4 ply, 4″×4″) were immediately placed on top of the nonwoven acquisition layer. A thin Plexiglas plate and a weight were placed on top of the gauze for one minute. The Plexiglas and weight exerted a total pressure of 0.25 psi. The gauze was weighed to determine the rewet result.

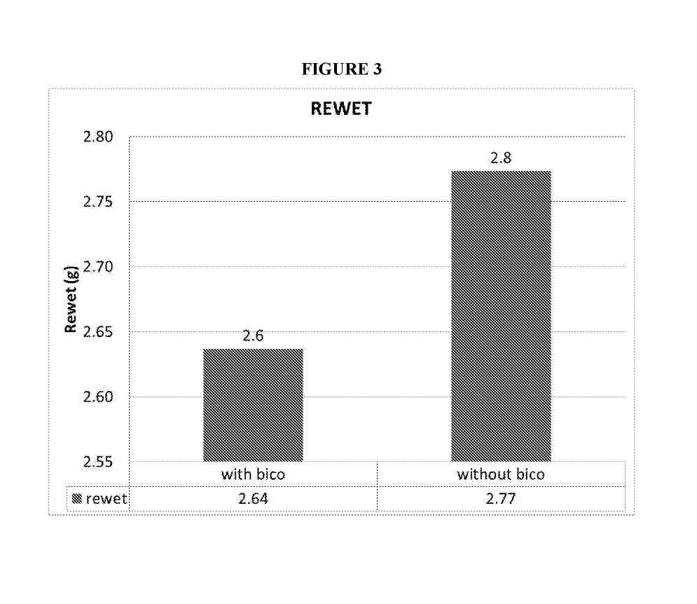

FIG. 3 illustrates the rewet results of each material. The rewet results are provided as a weight (g). The first material (i.e., the structure having a bicomponent fiber layer) showed improved liquid retention compared to the second material.

### Example 2

### Three-Layer Nonwoven Acquisition Material

The present Example provides a three-layer nonwoven acquisition material in accordance with the disclosed subject matter.

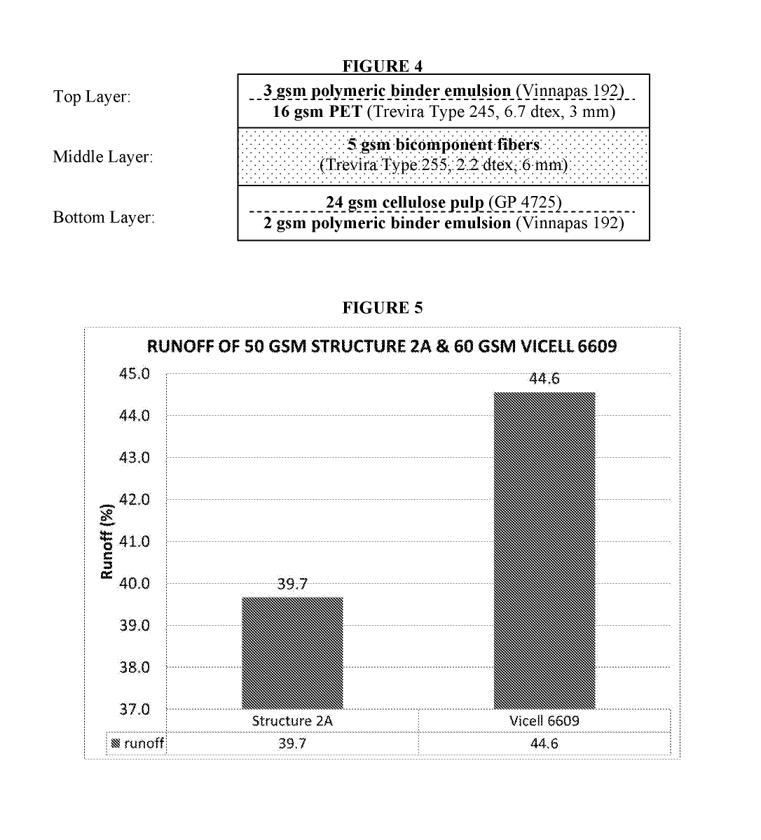

The material was formed using a lab padformer. The top layer of the three-layer, nonwoven acquisition material was composed of 16 gsm of PET fibers (Trevira Type 245, 6.7 dtex, 3 mm), which were bonded with a 3 gsm polymeric binder in the form of an emulsion (Vinnapas 192, Wacker). The middle layer was composed of 5 gsm bicomponent fibers (Trevira 1661, Type 255, 2.2 dtex, 6 mm). The bottom layer was composed of 24 gsm of cellulose (GP 4725, semi-treated pulp), which was bonded with a 2 gsm polymeric binder in the form of an emulsion (Vinnapas 192, Wacker). The average thickness of this structure was 1.02 mm. FIG. 4 gives a pictorial description of the acquisition material composition. Three samples of the same material were prepared.

The liquid acquisition characteristics of the acquisition material were measured with a synthetic blood solution. Synthetic blood was purchased from Johnson, Moen & Co. Inc. (Rochester, Minn.) (Lot #201141; February 2014). The synthetic blood had a surface tension of 40-44 dynes/cm (ASTM F23.40-F1670) and included various chemicals including ammonium polyacrylate polymer, Azo Red Dye, HPLC distilled water, among other proprietary ingredients. The synthetic blood was diluted with deionized water to a composition of 35% blood and 65% water.

The acquisition material was taped to a 45-degree plexiglass platform. 5 mL of synthetic blood (as measured in a 10 mL graduated cylinder) were poured rapidly onto the center of the acquisition material with the graduated cylinder approximately 1 cm from the surface of the acquisition material. The grams of synthetic blood runoff were recorded as the amount of the liquid which ran off the sample without being absorbed by it. As a comparator, a commercially available acquisition material, Vicell 6609 (LBAL, Georgia-Pacific, Steinfurt), was also tested under the same procedure.

FIG. 5 illustrates the percent of runoff from each material. FIG. 5 shows that, based on the averages of the samples, the lab-made nonwoven acquisition material yielded less runoff than the commercially available Vicell 6609 (LBAL, GP Steinfurt), despite having a lower basis weight.

### Example 3

### Liquid Acquisition Nonwoven Materials

The present Example provides two control liquid acquisition nonwoven materials for comparative purposes. These materials are designated 3A and 3B. Three sets of each material were prepared. Respectively, these controls are commercially available products: an LBAL (latex-bonded airlaid) product (Vicell 6609, also called 60 MAR S II) and an MBAL (multi-bonded airlaid) (Vizorb 3074, also referenced as 60 MBAL), both products made by Georgia-Pacific in Steinfurt, Germany. Both control products have a basis weight of 60 gsm.

The liquid acquisition characteristics of the control materials were measured with a synthetic blood solution using the liquid acquisition performance testing procedures described below. Synthetic blood was purchased from Johnson, Moen & Co. Inc. (Rochester, Minn.) (Lot #201141; February 2014). The synthetic blood had a surface tension of 40-44 dynes/cm (ASTM F23.40-F1670) and included various chemicals including ammonium polyacrylate polymer, Azo Red Dye, HPLC distilled water, among other proprietary ingredients. The synthetic blood was diluted with deionized water to a composition of 35% blood and 65% water.

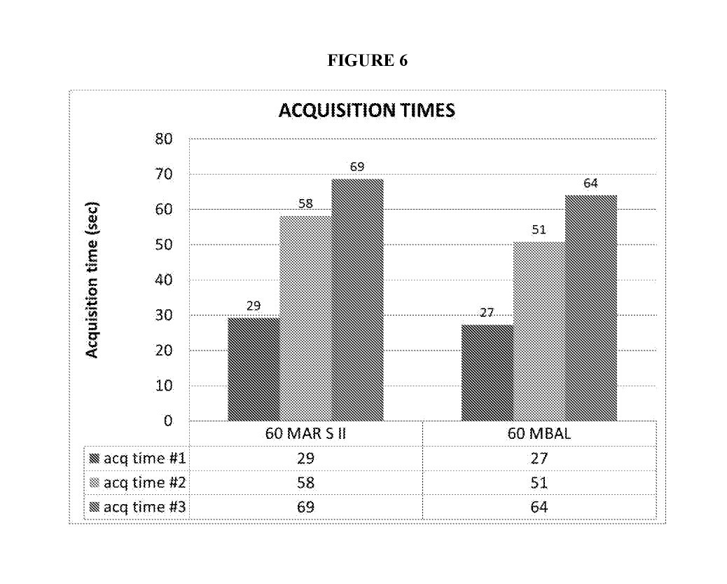

The MAR S II product was placed on top of the commercially available nonwoven core material (175 MBS3A, Georgia-Pacific, Steinfurt, Germany) to form an absorbent composite. This composite was compressed with an 8.190 kg plate for 1 minute. The prepared composite was tested for its liquid acquisition performance using the prepared synthetic blood solution. The composite was insulted with 4 mL of the synthetic blood at a rate of 10 mL/min. After completing the insult, the acquisition time was measured. A total of three insults were performed, yielding acquisition times, #1, #2, and #3. The time interval between the insults was 10 minutes. The preceding steps were repeated for the MBAL product. FIG. 6 illustrates the average acquisition times of the two products for each of the three insults.

### Example 4

### Nonwoven Structures with Cellulose Fibers

The present Example provides for various structures (Structures 4A-4J) with cellulose fibers in the bottom layers of the materials.

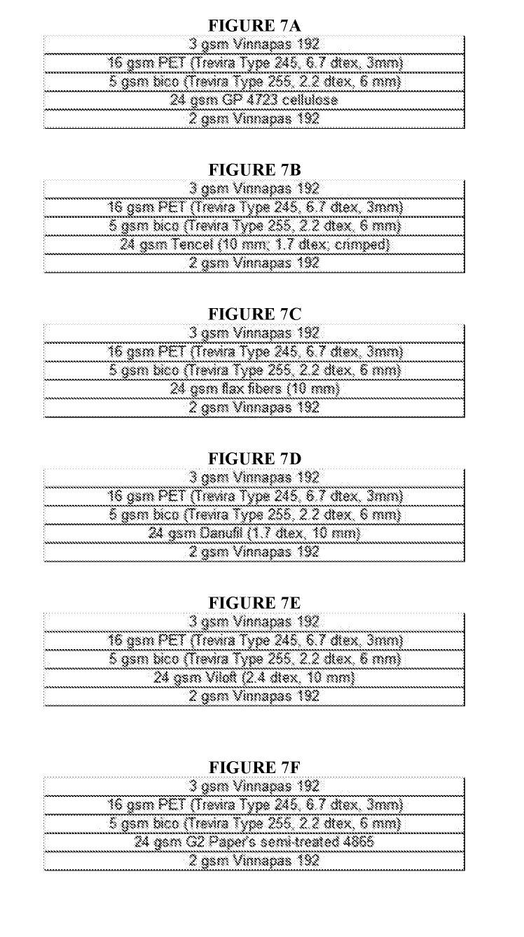

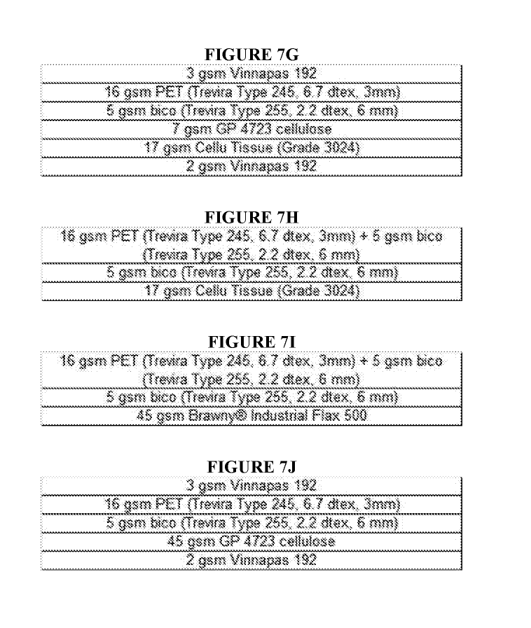

Structure 4A is a three-layer airlaid nonwoven structure which can be formed using a lab padformer. The top layer of Structure 4A was composed of 16 gsm of PET fibers (Trevira Type 245, 6.7 dtex, 3 mm), which were bonded with a 3 gsm polymeric binder sprayed on the airlaid web in the form of emulsion (Vinnapas 192, Wacker). The middle layer was composed of 5 gsm bicomponent fibers (Trevira Type 255, 2.2 dtex, 6 mm). The bottom layer was composed of 24 gsm of cellulose (GP 4723, fully treated pulp made by Georgia-Pacific), which was bonded with a 2 gsm polymeric binder in the form of emulsion (Vinnapas 192, Wacker). Two samples of the same structure were prepared. The average thickness of this structure was 1.01 mm. FIG. 7A gives a pictorial description of Structure 4A and its composition. Structure 4B is a three-layer airlaid nonwoven structure which can be formed using a lab padformer. The top layer of the three-layer Structure 4B was composed of 16 gsm of PET fibers (Trevira Type 245, 6.7 dtex, 3 mm), which were bonded with a 3 gsm polymeric binder sprayed on the airlaid web in the form of emulsion (Vinnapas 192, Wacker). The middle layer was composed of 5 gsm bicomponent fibers (Trevira Type 255, 2.2 dtex, 6 mm). The bottom layer was composed of 24 gsm of cellulose (Tencel, 10 mm, 1.7 dtex, crimped, made by Lenzing), which was bonded with a 2 gsm polymeric binder in the form of emulsion (Vinnapas 192, Wacker). Two samples of the same structure were prepared. The average thickness of this structure was 1.13 mm. FIG. 7B gives a pictorial description of Structure 4B its composition.

Structure 4C is a three-layer airlaid nonwoven structure which can be formed using a lab padformer. The top layer of the three-layer Structure 4C was composed of 16 gsm of PET fibers (Trevira Type 245, 6.7 dtex, 3 mm), which were bonded with a 3 gsm polymeric binder sprayed on the airlaid web in the form of emulsion (Vinnapas 192, Wacker). The middle layer was composed of 5 gsm bicomponent fibers (Trevira Type 255, 2.2 dtex, 6 mm). The bottom layer was composed of 24 gsm of cellulose flax fibers (cut to 10 mm length) which were bonded with a 2 gsm polymeric binder sprayed on the airlaid web in the form of emulsion (Vinnapas 192, Wacker). Three samples of the same structure were prepared. The average thickness of this structure was 0.87 mm. FIG. 7C gives a pictorial description of Structure 4C and its composition.

Structure 4D is a three-layer airlaid nonwoven structure which can be formed using a lab padformer. The top layer of the three-layer Structure 4D was composed of 16 gsm of PET fibers (Trevira Type 245, 6.7 dtex, 3 mm), which were bonded with a 3 gsm polymeric binder sprayed on the airlaid web in the form of emulsion (Vinnapas 192, Wacker). The middle layer was composed of 5 gsm bicomponent fibers (Trevira Type 255, 2.2 dtex, 6 mm). The bottom layer was composed of 24 gsm of cellulose (Danufil, 1.7 dtex, 10 mm made by Kelheim), which was bonded with a 2 gsm polymeric binder in the form of emulsion (Vinnapas 192, Wacker). Two samples of the same structure were prepared. The average thickness of this structure was 1.02 mm. FIG. 7D gives a pictorial description of Structure 4D and its composition.

Structure 4E is a three-layer airlaid nonwoven structure which can be formed using a lab padformer. The top layer of the three-layer structure 4E was composed of 16 gsm of PET fibers (Trevira Type 245, 6.7 dtex, 3 mm), which were bonded with a 3 gsm polymeric binder sprayed on the airlaid web in the form of emulsion (Vinnapas 192, Wacker). The middle layer was composed of 5 gsm bicomponent fibers (Trevira Type 255, 2.2 dtex, 6 mm). The bottom layer was composed of 24 gsm of cellulose fibers (Viloft, 2.4 dtex, 10 mm, made by Kelheim), which was bonded with a 2 gsm polymeric binder in the form of emulsion (Vinnapas 192, Wacker). Three samples of the same structure were prepared. The average thickness of this structure was 1.16 mm. FIG. 7E gives a pictorial description of Structure 4E and its composition.

Structure 4F is a three-layer airlaid nonwoven structure which can be formed using a lab padformer. The top layer of the three-layer Structure 4F was composed of 16 gsm of PET fibers (Trevira Type 245, 6.7 dtex, 3 mm), which were bonded with a 3 gsm polymeric binder sprayed on the airlaid web in the form of emulsion (Vinnapas 192, Wacker). The middle layer was composed of 5 gsm bicomponent fibers (Trevira Type 255, 2.2 dtex, 6 mm). The bottom layer was composed of 24 gsm of odor control cellulose fiber (G2 Paper's semi-treated 4865 made by Georgia-Pacific), which was bonded with a 2 gsm polymeric binder in the form of emulsion (Vinnapas 192, Wacker). Three samples of the same structure were prepared. The average thickness of this structure was 0.95 mm. FIG. 7F gives a pictorial description of Structure 4F and its composition.

Structure 4G is a four-layer nonwoven structure which can be formed using a lab padformer. The top layer of the four-layer Structure 4G was composed of 16 gsm of PET fibers (Trevira Type 245, 6.7 dtex, 3 mm), which were bonded with a 3 gsm polymeric binder sprayed on the airlaid web in the form of emulsion (Vinnapas 192, Wacker). Underneath this PET layer is a layer composed of 5 gsm bicomponent fibers (Trevira Type 255, 2.2 dtex, 6 mm). Below the bicomponent fiber layer is 7 gsm of cellulose (GP 4723, fully treated pulp made by Georgia-Pacific). The bottom layer was composed of 17 gsm of cellulose (Grade 3024 Cellu Tissue made by Clearwater), which was bonded with a 2 gsm polymeric binder in the form of emulsion (Vinnapas 192, Wacker). Two samples of the same structure were prepared. The average thickness of this structure was 1.14 mm. FIG. 7G gives a pictorial description of Structure 4G and its composition.

Structure 4H is a three-layer airlaid nonwoven structure which can be formed using a lab padformer. This structure is similar to the structure in FIG. 7G, except that the 7 gsm of GP 4723 cellulose is omitted from the structure. Also, no polymeric binder was sprayed onto the surfaces of both sides. The top layer of the three-layer, nonwoven acquisition layer of structure 4H was composed of a homogenous mixture of 16 gsm of PET fibers (Trevira Type 245, 6.7 dtex, 3 mm) and 5 gsm bicomponent fibers (Trevira Type 255, 2.2 dtex, 6 mm). The middle layer was composed of 5 gsm bicomponent fibers (Trevira Type 255, 2.2 dtex, 6 mm). The bottom layer was composed of 17 gsm of cellulose (Grade 3024 Cellu Tissue). Three samples of the same structure were prepared. The average thickness of this structure was 0.77 mm. FIG. 7H gives a pictorial description of Structure 4H and its composition.

Structure 4I is a three-layer airlaid nonwoven structure which can be formed using a lab padformer. The top layer of the three-layer Structure 4I was composed of a homogeneous mixture of 16 gsm of PET fibers (Trevira Type 245, 6.7 dtex, 3 mm) and 5 gsm of bicomponent fibers (Trevira Type 255, 2.2 dtex, 6 mm). No polymeric binder was applied to the surface of this top layer. The middle layer was composed of 5 gsm bicomponent fibers (Trevira Type 255, 2.2 dtex, 6 mm). The bottom layer was composed of 45 gsm of cellulose (Brawny® Industrial Flax 500 made by Georgia-Pacific). No polymeric binder was applied to the surface of the Brawny® Industrial Flax 500. Two samples of the same structure were prepared. The average thickness of this structure was 0.92 mm. FIG. 71 gives a pictorial description of Structure 4I and its composition.

Structure 4J is a three-layer airlaid nonwoven structure which can be formed using a lab padformer. The top layer of the three-layer Structure 4J was composed of 16 gsm of PET fibers (Trevira Type 245, 6.7 dtex, 3 mm), which were bonded with a 3 gsm polymeric binder sprayed on the airlaid web in the form of emulsion (Vinnapas 192, Wacker). The middle layer was composed of 5 gsm bicomponent fibers (Trevira Type 255, 2.2 dtex, 6 mm). The bottom layer was composed of 45 gsm of cellulose (GP 4723, fully treated pulp from Leaf River), which was bonded with a 2 gsm polymeric binder in the form of emulsion (Vinnapas 192, Wacker). Three samples of the same structure were prepared. The average thickness of this structure was 1.30 mm. FIG. 7J gives a pictorial description of Structure 4J and its composition.

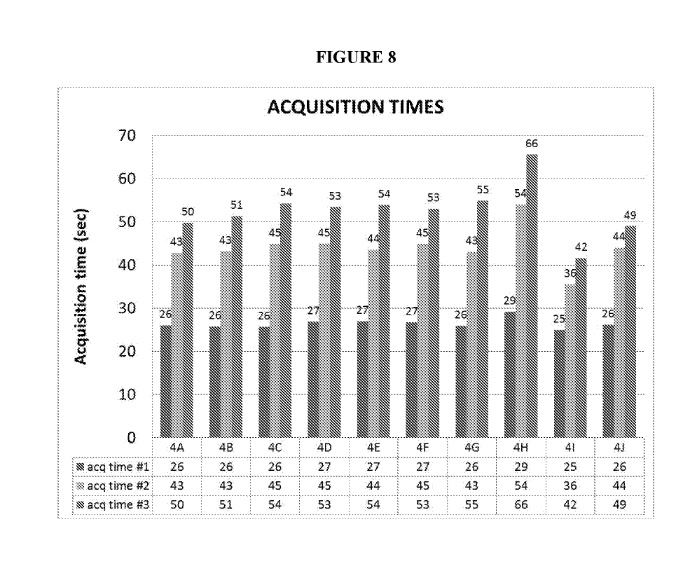

Structures 4A-4J were tested for liquid acquisition characteristics. The measurements were conducted according to the procedures described in Example 3. FIG. 8 is a summary of the average acquisition times of each structure for each of the three insults.

### Example 5

### Nonwoven Structures with Bicomponent Fibers

The present Example provides for various structures (Structures 5A-5C) with bicomponent fibers in the middle layer of the materials.

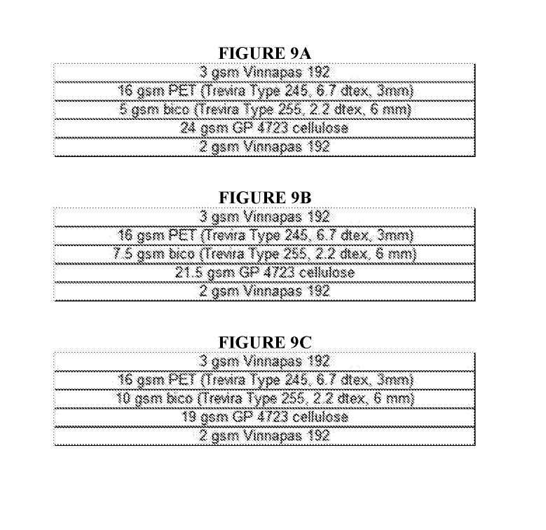

Structure 5A is a three-layer airlaid nonwoven structure which can be formed using a lab padformer. The top layer of the three-layer Structure 5A was composed of 16 gsm of PET fibers (Trevira Type 245, 6.7 dtex, 3 mm), which were bonded with a 3 gsm polymeric binder sprayed on the airlaid web in the form of emulsion (Vinnapas 192, Wacker). The middle layer was composed of 5 gsm bicomponent fibers (Trevira Type 255, 2.2 dtex, 6 mm). The bottom layer was composed of 24 gsm of cellulose (GP 4723, fully treated pulp made by Georgia-Pacific), which was bonded with a 2 gsm polymeric binder in the form of emulsion (Vinnapas 192, Wacker). Two samples of the same structure were prepared. The average thickness of this structure was 1.01 mm. FIG. 9A gives a pictorial description of Structure 5A and its composition.

Structure 5B is a three-layer airlaid nonwoven structure which can be formed using a lab padformer. The top layer of the three-layer Structure 5B was composed of 16 gsm of PET fibers (Trevira Type 245, 6.7 dtex, 3 mm), which were bonded with a 3 gsm polymeric binder sprayed on the airlaid web in the form of emulsion (Vinnapas 192, Wacker). The middle layer was composed of 7.5 gsm bicomponent fibers (Trevira Type 255, 2.2 dtex, 6 mm). The bottom layer was composed of 21.5 gsm of cellulose (GP 4723, fully treated pulp made by Georgia-Pacific), which was bonded with a 2 gsm polymeric binder in the form of emulsion (Vinnapas 192, Wacker). Three samples of the same structure were prepared. The average thickness of this structure was 1.02 mm. FIG. 9B gives a pictorial description of Structure 5B and its composition.

Structure 5C is a three-layer airlaid nonwoven structure which can be formed using a lab padformer. The top layer of the three-layer Structure 5C was composed of 16 gsm of PET fibers (Trevira Type 245, 6.7 dtex, 3 mm), which were bonded with a 3 gsm polymeric binder sprayed on the airlaid web in the form of emulsion (Vinnapas 192, Wacker). The middle layer was composed of 10 gsm bicomponent fibers (Trevira Type 255, 2.2 dtex, 6 mm). The bottom layer was composed of 19 gsm of cellulose (GP 4723, fully treated pulp made by Georgia-Pacific), which was bonded with a 2 gsm polymeric binder in the form of emulsion (Vinnapas 192, Wacker). Two samples of the same structure were prepared. The average thickness of this structure was 1.04 mm. FIG. 9C gives a pictorial description of Structure 5C and its composition.

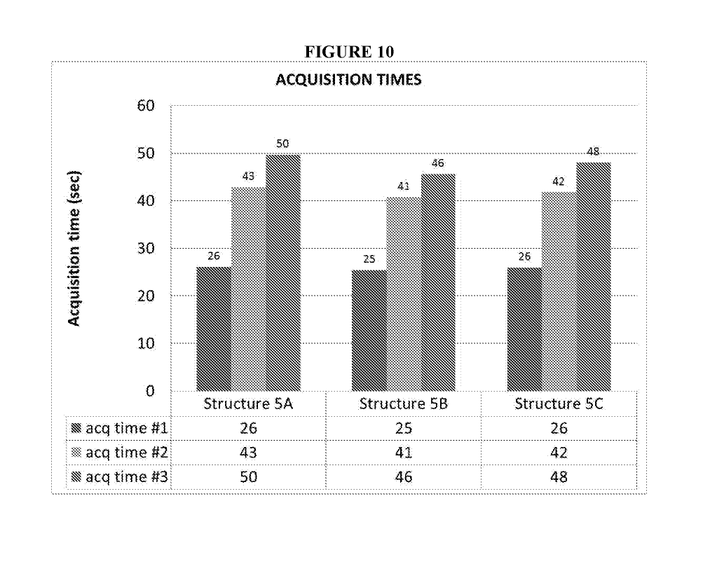

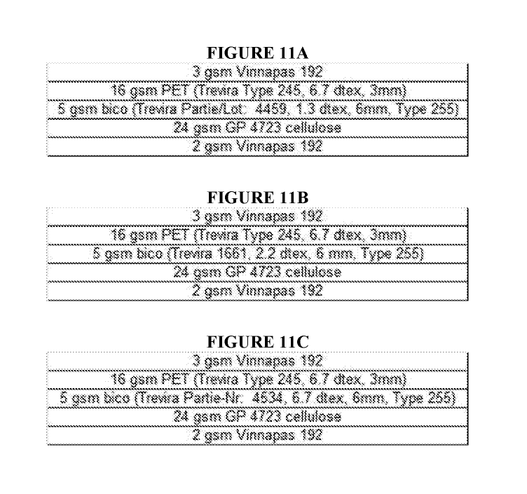

Structures 5A-5C were tested for their liquid acquisition characteristics in the same way as describes in Example 3. FIG. 10 summarizes the average acquisition times of these structures for each of the three insults.

### Example 6

### Nonwoven Structures with Bicomponent Fibers

The present Example provides for various Structures (Structures 6A-6C) with bicomponent fibers having various dtex numbers.

Structure 6A is a three-layer airlaid nonwoven structure which can be formed using a lab padformer. The top layer of the three-layer Structure 6A was composed of 16 gsm of PET fibers (Trevira Type 245, 6.7 dtex, 3 mm), which were bonded with a 3 gsm polymeric binder sprayed on the airlaid web in the form of emulsion (Vinnapas 192, Wacker). The middle layer was composed of 5 gsm bicomponent fibers (Trevira Partie/Lot: 4459, 1.3 dtex, 6mm, Type 255). The bottom layer was composed of 24 gsm of cellulose (GP 4723, fully treated pulp made by Georgia-Pacific), which was bonded with a 2 gsm polymeric binder in the form of emulsion (Vinnapas 192, Wacker). Two samples of the same structure were prepared. The average thickness of this structure was 1.01 mm. FIG. 11A gives a pictorial description of Structure 6A and its composition.

Structure 6B is a three-layer airlaid nonwoven structure which can be formed using a lab padformer. The top layer of the three-layer Structure 6B was composed of 16 gsm of PET fibers (Trevira Type 245, 6.7 dtex, 3 mm), which were bonded with a 3 gsm polymeric binder sprayed on the airlaid web in the form of emulsion (Vinnapas 192, Wacker). The middle layer was composed of 5 gsm bicomponent fibers (Trevira 1661, 2.2 dtex, 6 mm, Type 255). The bottom layer was composed of 24 gsm of cellulose (GP 4723, fully treated pulp made by Georgia-Pacific), which was bonded with a 2 gsm polymeric binder in the form of emulsion (Vinnapas 192, Wacker). Two samples of the same structure were prepared. The average thickness of this structure was 1.01 mm. FIG. 11B gives a pictorial description of Structure 6B and its composition.

Structure 6C is a three-layer airlaid nonwoven structure which can be formed using a lab padformer. The top layer of the three-layer, nonwoven acquisition layer of Structure 6C was composed of 16 gsm of PET fibers (Trevira Type 245, 6.7 dtex, 3 mm), which were bonded with a 3 gsm polymeric binder in the form of emulsion (Vinnapas 192, Wacker). The middle layer was composed of 5 gsm bicomponent fibers (Trevira Partie-Nr: 4534, 6.7 dtex, 6mm, Type 255). The bottom layer was composed of 24 gsm of cellulose (GP 4723, fully treated pulp from Leaf River), which was bonded with a 2 gsm polymeric binder in the form of emulsion (Vinnapas 192, Wacker). Two samples of the same structure were prepared. The average thickness of this structure was 1.02 mm. FIG. 11C gives a pictorial description of Structure 6C and its composition.

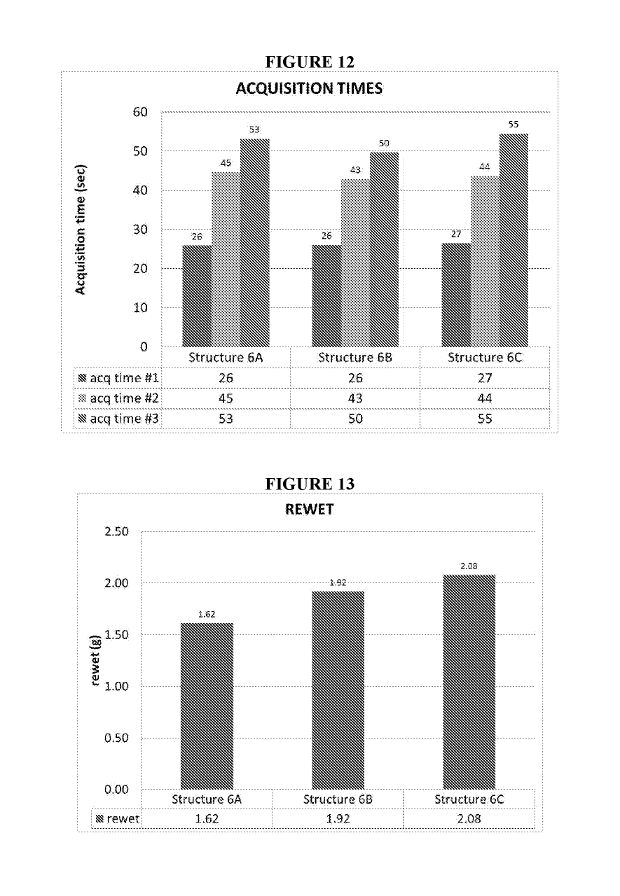

The acquisition characteristics of Structures 6A-6C were measured as described in Example 3. FIG. 12 summarizes the average acquisition times of these structures for each of the three insults.

Additionally, the rewet characteristics of Structures 6A-6C were analyzed after measuring the three acquisition times. Three pieces of gauze (Covidien's Curity, all-purpose sponges, non-woven, 4 ply, 4″×4″) were immediately placed on top of the structures. A thin Plexiglas plate and a weight were placed on top of the gauze for one minute. The Plexiglas and weight exerted a total pressure of 0.25 psi. The gauze was weighed to determine the rewet result (i.e., the difference between the weight of the gauze after the test and the weight of the gauze before the test). FIG. 13 illustrates the rewet results of Structures 6A-6C. The rewet results are provided as a weight (g). Structure 6A, which contained the finest (lowest dtex) bicomponent fibers in its middle layer, released the least moisture during the test. These data show that using finer bicomponent fibers in the middle layer can lead to improved rewet characteristics.

### Example 7

### Nonwoven Structures with Bicomponent Fibers

The present Example provides for various structures (Structures 7A and 7B) with two types of PET fiber in the upper layer of the structures.

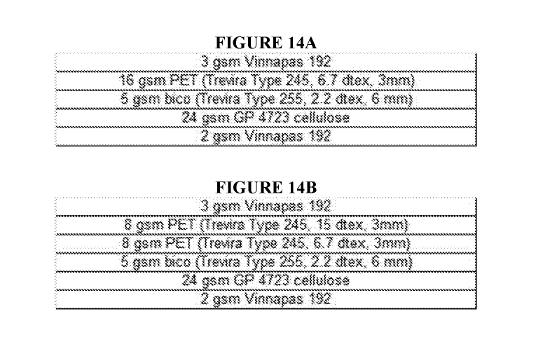

Structure 7A is a three-layer airlaid nonwoven structure which can be formed using a lab padformer. The top layer of the three-layer Structure 7A was composed of 16 gsm of PET fibers (Trevira Type 245, 6.7 dtex, 3 mm), which were bonded with a 3 gsm polymeric binder sprayed on the web in the form of emulsion (Vinnapas 192, Wacker). The middle layer was composed of 5 gsm bicomponent fibers (Trevira Type 255, 2.2 dtex, 6 mm). The bottom layer was composed of 24 gsm of cellulose (GP 4723, fully treated pulp made by Georgia-Pacific), which was bonded with a 2 gsm polymeric binder in the form of emulsion (Vinnapas 192, Wacker). Two samples of the same structure were prepared. The average thickness of this structure was 1.01 mm. FIG. 14A gives a pictorial description of Structure 7A and its composition.

Structure 7B is a four-layer airlaid nonwoven structure which can be formed using a lab padformer. The top layer of the four-layer, nonwoven acquisition layer of Structure 7B was composed of 8 gsm of PET fibers (Trevira Type 245, 15 dtex, 3 mm). Underneath this layer is another PET fiber layer but of a lower dtex. This second layer was composed of 8 gsm of PET fibers (Trevira Type 245, 6.7 dtex, 3 mm). Both PET fiber layers were bonded with a 3 gsm polymeric binder sprayed on the airlaid web in the form of emulsion (Vinnapas 192, Wacker). Below the two PET fiber layer is 5 gsm bicomponent fibers (Trevira Type 255, 2.2 dtex, 6 mm). The bottom layer was composed of 24 gsm of cellulose (GP 4723, fully treated pulp made by Georgia-Pacific), which was bonded with a 2 gsm polymeric binder in the form of emulsion (Vinnapas 192, Wacker). Three samples of the same structure were prepared. The average thickness of this structure was 0.99 mm. FIG. 14B gives a pictorial description of Structure 7B and its composition.

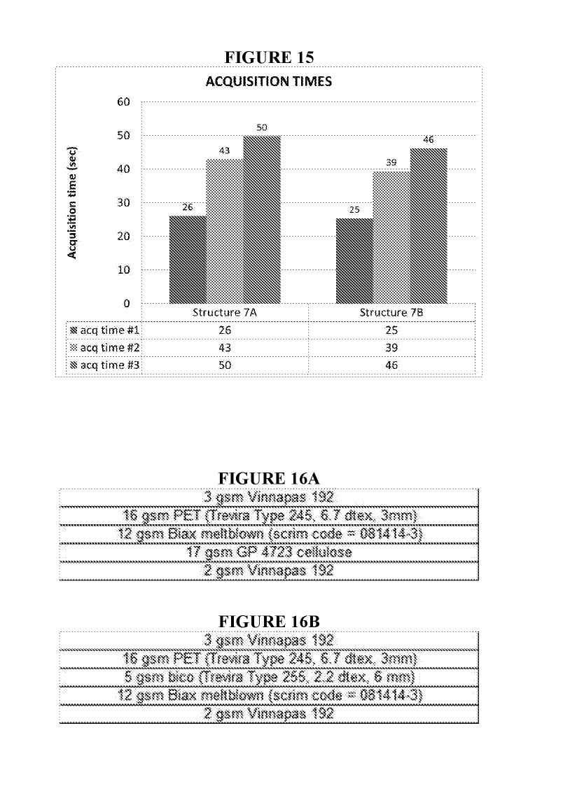

Structures 7A and 7B were tested for liquid acquisition characteristics according to the method described in Example 3. FIG. 15 summarizes the average acquisition times of Structures 7A and 7B for each of the three insults.

### Example 8

### Nonwoven Structures with Bonded Synthetic Filaments

The present Example provides for various structures (Structures 8A and 8B) with a layer made of bonded synthetic filaments.

Structure 8A is a three-layer airlaid nonwoven structure which can be formed using a lab padformer. The top layer of the three-layer Structure 8A was composed of 16 gsm of PET fibers (Trevira Type 245, 6.7 dtex, 3 mm), which were bonded with a 3 gsm polymeric binder sprayed on the airlaid web in the form of emulsion (Vinnapas 192, Wacker). The middle layer was composed of a 12 gsm meltblown polypropylene layer (made by Biax). The bottom layer was composed of 17 gsm of cellulose (GP 4723, fully treated pulp from Leaf River), which was bonded with a 2 gsm polymeric binder in the form of emulsion (Vinnapas 192, Wacker). Two samples of the same structure were prepared. The average thickness of this structure was 1.04 mm. FIG. 16A gives a pictorial description of Structure 8A and its composition.

Structure 8B is a three-layer airlaid nonwoven structure which can be formed using a lab padformer. The top layer of the three-layer Structure 8B was composed of 16 gsm of PET fibers (Trevira Type 245, 6.7 dtex, 3 mm), which was bonded with a 3 gsm polymeric binder sprayed on the airlaid web in the form of emulsion (Vinnapas 192, Wacker). The middle layer was composed of 5 gsm bicomponent fibers (Trevira Type 255, 2.2 dtex, 6 mm). The bottom layer was composed of a 12 gsm meltblown polypropylene layer, which was coated with a 2 gsm polymeric binder in the form of emulsion (Vinnapas 192, Wacker). Three samples of the same structure were prepared. The average thickness of this structure was 0.85 mm. FIG. 16B gives a pictorial description of Structure 8B and its composition.

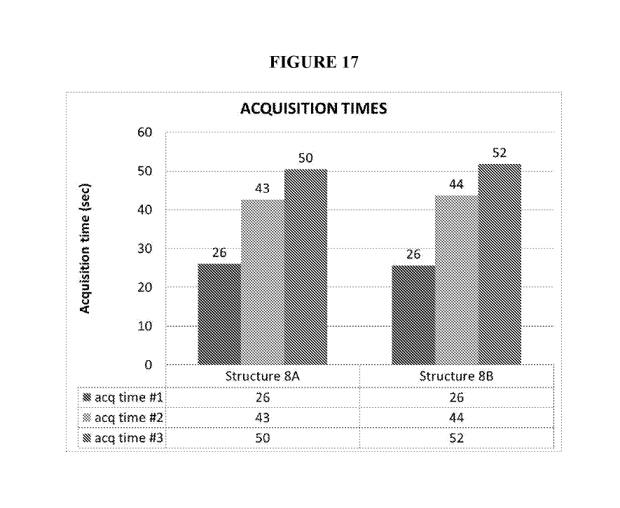

Structures 8A and 8B were tested for liquid acquisition characteristics according to the method described in Example 3. FIG. 17 shows the results of the acquisition times for Structures 8A and 8B for each of the three insults.

### Example 9

### 4-Layer Nonwoven Structures

The present Example provides for various structures (Structures 9A-9C) with four distinct layers.

All three structures have four distinct layers as follows. The first top layer is composed of PET Fibers (Trevira Type 245, 6.7 dtex, 3mm), which are bonded with a polymeric binder sprayed on the airlaid web in the form of emulsion (Vinnapas 192, Wacker). The second layer, adjacent to the first top layer, is composed of bicomponent fibers. The third layer, adjacent to the second layer is composed of a mixture of pulp (GP4723) and bicomponent fibers. The fourth and final layer, which is below third layer, is composed of cellulose pulp (GP4723), which is bonded with a polymeric binder sprayed on the airlaid web in the form of emulsion (Vinnapas 192, Wacker).

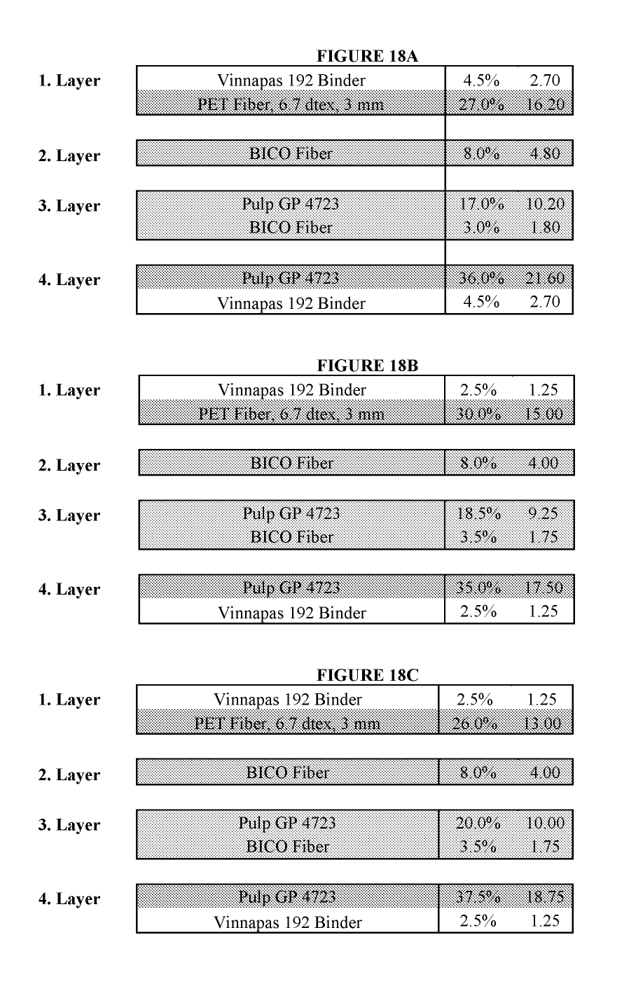

FIGS. 18A-18C give a pictorial description of the layers and content of the structures. FIG. 18A depicts Structure 9A, which is a 60 gsm material. FIG. 18B depicts Structure 9B, which is a 50 gsm material. FIG. 18C depicts Structure 9C, which is also a 50 gsm material.

### Example 10

### Two-Layer Nonwoven Structures

The present Example provides for two experimental structures (Structures 10A and 10B), each composed of a bicomponent fiber top layer and a bottom layer. Both structures were made using a lab padformer and cured for 5 minutes in a lab through-air-dry oven.

The top layer of the two-layer Structure 10A was composed of 23 gsm of eccentric bicomponent fibers (5.7 dtex, 4 mm, made by FiberVisions) and the bottom layer was composed of 17 gsm of cellulose tissue (Grade 3024 Cellu Tissue made by Clearwater). Three samples of the same structure were prepared. The average thickness of this structure was 1.9 mm.

The top layer of the two-layer Structure 10B was composed of 28 gsm of eccentric bicomponent fibers (5.7 dtex, 4 mm, made by FiberVisions) and the bottom layer was composed of a 12 gsm bonded polypropylene filaments (made by Biax). Three samples of the same structure were prepared. The average thickness of this structure was 2.0 mm.

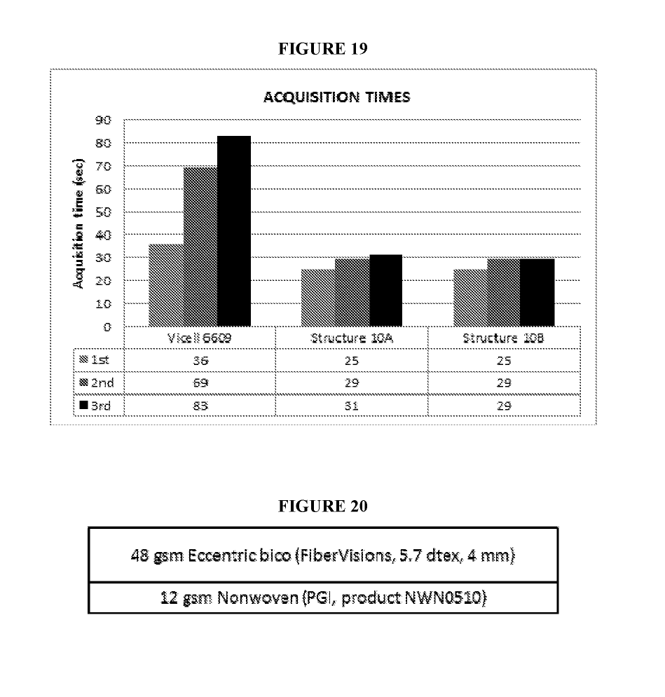

Structures 10A and 10B were tested for liquid acquisition characteristics according to the method described in Example 3. FIG. 19 summarizes the average acquisition times of Structures 10A and 10B for each of the three insults. For comparison the results for the control LBAL (latex-bonded airlaid) product Vicell 6609 (Georgia-Pacific, Steinfurt, Germany) are shown in FIG. 19 as well.

### Example 11

### Two-Layer Nonwoven Structure

The present Example provides for an experimental nonwoven structure (Structure 11A). The nonwoven structure was made on a pilot-scale drum-forming airlaid line.

FIG. 20 depicts Structure 11A. The top layer of the structure was composed of 48 gsm of eccentric bicomponent fibers (5.7 dtex, 4 mm, made by FiberVisions) and the bottom layer was composed of 12 gsm of synthetic nonwoven material (NWN0510 made by PGI).

A sample of Structure 11A was tested for liquid acquisition performance and rewet characteristics in a commercially available Major Brand Baby Diaper (MBBD). The MBBD product contains a topsheet layer and a synthetic high-loft nonwoven material serving as a fluid acquisition layer. Its measured basis weight was about 80 gsm and it had a rectangular shape with a length of about 24.2 cm and a width of about 8.6 cm. The MBBD product was trimmed along all four edges. The MBBD product was then placed in an oven for 5 minutes at 100° C. After 5 minutes, the topsheet layer was peeled off the high-loft acquisition layer. The high-loft acquisition layer was then separated from the diaper and placed back in the original position. The topsheet was subsequently placed back on the high-loft acquisition layer.

To confirm that the performance of the reassembled MBBD product was comparable to the original MBBD product “as is” without disassembling and reassembling, both the reassembled product and original product were tested. Comparable results were obtained from both products, indicating that the disassembling and reassembling procedures do not have a significant impact on the performance of the original product. Therefore, a reassembled product containing Structure 11A can be compared to the original MBBD product.

To evaluate the effect of Structure 11A on the performance of the MBBD product, the original high-loft acquisition layer of the MBBD product was replaced with Structure 11A cut to the same dimensions as the original high-loft acquisition layer. The top layer of Structure 11A (i.e., the layer containing eccentric bicomponent) was oriented towards the top side of the modified MBBD product.

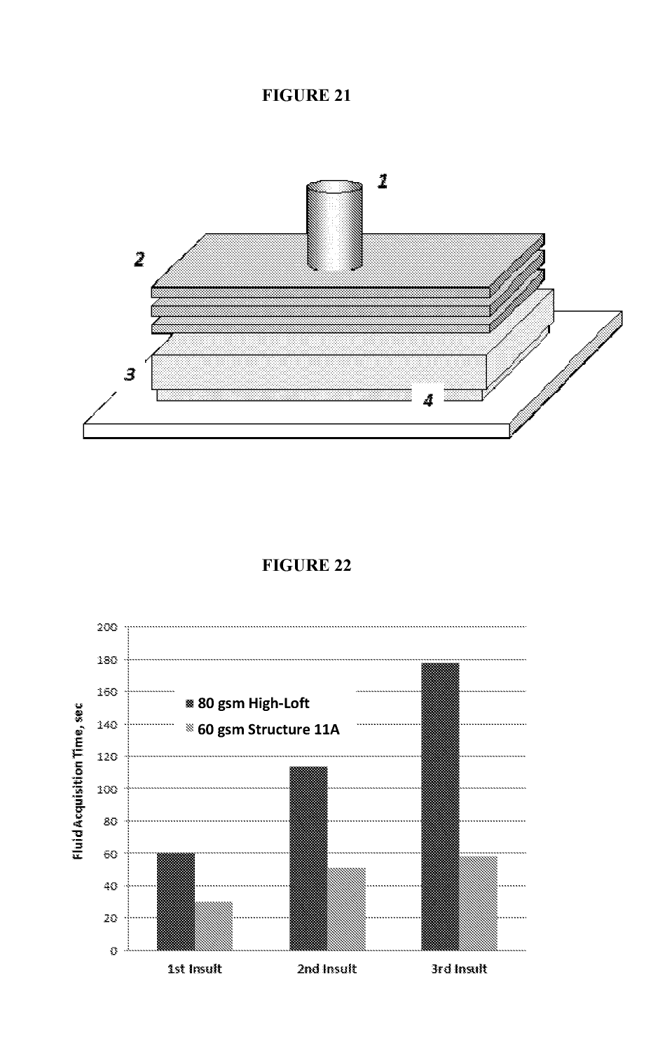

FIG. 21 depicts the testing apparatus. The absorbent product to be tested 4 was covered with a piece of soft foam 3 and metal-plate weights 2 exerting a pressure of about 2.8 kPa on the product. A cylinder 1 was used to insult the product with a 0.9% solution of sodium chloride containing a blue dye. The cylinder had an inner diameter of 3.8 cm. The MBBD product containing the original high-loft acquisition layer and the MBBD product containing Structure 11A were each insulted three times with 75 mL of the sodium chloride solution at a rate of 7 mL/min using a pump. The interval time between the insults was 20 minutes. The idle time after the third insult was also 20 minutes. After 20 minutes, the foam, metal-plate weights, and cylinder were removed.

FIG. 22 illustrates the acquisition times of the two MBBD products for each of the three insults. The MBBD product containing Structure 11A showed improved acquisition times compared to the original MBBD product.

Further, the rewet characteristics of both MBBD products were analyzed after measuring the acquisition times. Eight pre-weighed sheets of Coffi collagen sheets (Viscofan) were cut to be 23.5 cm×10.2 cm and placed on top of the MBBD product containing the original high-loft acquisition layer and the MBBD product containing Structure 11A. The foam, metal-plate weights, and cylinder were replaced on top of the Coffi collagen sheets. After five minutes, the Coffi collagen sheets were removed and weighed to determine the rewet result.

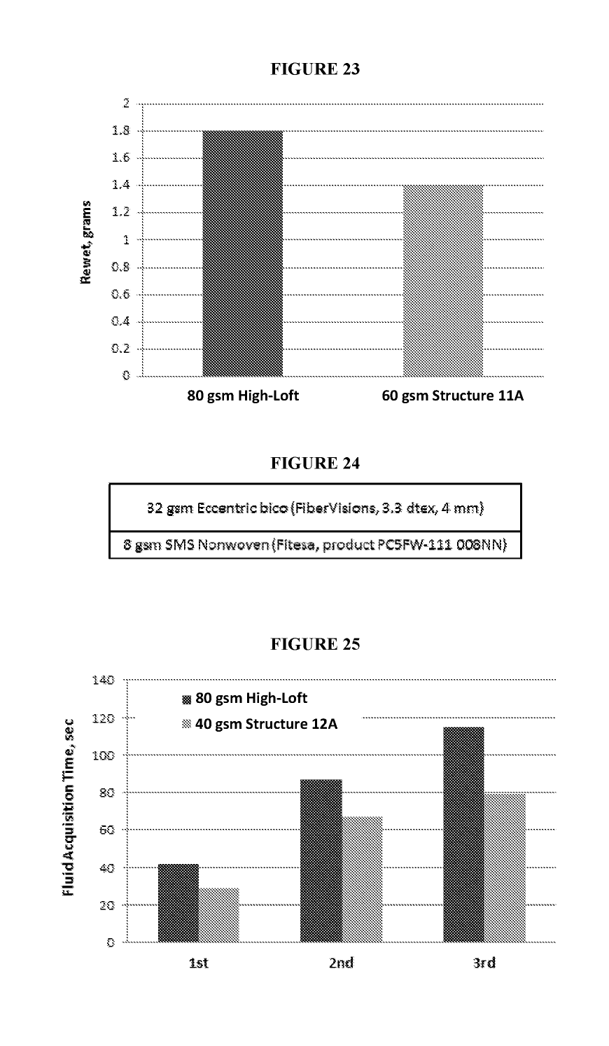

FIG. 23 illustrates the rewet results of each MBBD product. The rewet results are provided as a weight (grams). The MBBD product containing Structure 11A showed improved liquid retention compared to the original MBBD product. These data suggest that Structure 11A has improved liquid acquisition performance and rewet characteristics compared to the original high-loft acquisition layer of the MBBD product.

### Example 12

### Two-Layer Nonwoven Structure

The present Example provides for an experimental nonwoven structure (Structure 12A).

FIG. 24 depicts Structure 12A. The bottom layer of the structure was composed of 8 gsm of a hydrophobic spunbond-meltblown-spunbond (SMS) nonwoven (Fitesa Germany GmbH, product code PC5FW-111 008NN). The top layer was formed using lab pad-forming equipment and was composed of 32 gsm of eccentric bicomponent fibers (3.3 dtex, 4 mm, made by FiberVisions). The structure was compacted and then placed in a through-air oven for 4 minutes at 138° C.

A sample of Structure 12A was tested for liquid acquisition performance and rewet characteristics in a commercial Major Brand Baby Diaper (MBBD) as described in Example 11. To evaluate the effect of Structure 12A on the performance of the MBBD product, the original high-loft acquisition layer of the MBBD product was replaced with Structure 12A cut to the same dimensions as the original high-loft acquisition layer. The top layer of Structure 12A (i.e., the layer containing eccentric bicomponent) was oriented towards the top side of the modified MBBD product.

Both the MBBD product containing the original high-loft acquisition layer and the MBBD product containing Structure 12A were tested for liquid acquisition performance and rewet characteristics as described in Example 11 and using the testing apparatus depicted in FIG. 21.

FIG. 25 illustrates the acquisition times of the two MBBD products for each of the three insults. The MBBD product containing Structure 12A showed improved acquisition times compared to the original MBBD product.

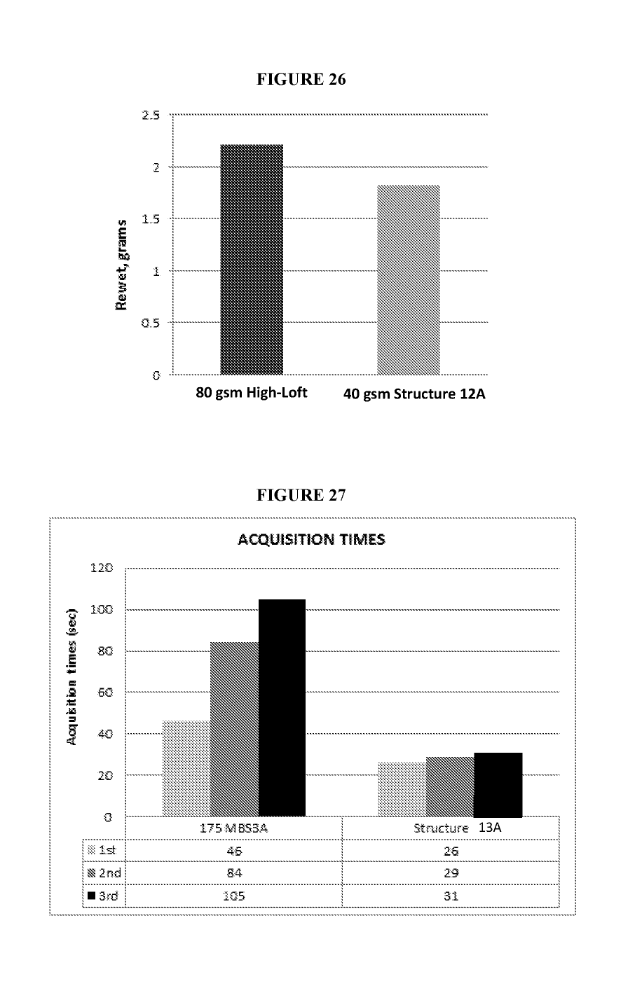

FIG. 26 illustrates the rewet results of each MBBD product. The rewet results are provided as a weight (grams). The MBBD product containing Structure 12A showed improved liquid retention compared to the original MBBD product. These data suggest that Structure 12A has improved liquid acquisition performance and rewet characteristics compared to the original high-loft acquisition layer of the MBBD product.

### Example 13

### Nonwoven Structure Containing Superabsorbent Polymer Powder

The present Example provides for an airlaid experimental structure (Structure 13A) containing superabsorbent polymer powder.

Structure 13A is composed of a layer of 18 gsm of eccentric bicomponent fibers (5.7 dtex, 4 mm, made by FiberVisions) airlaid on the absorbent nonwoven core having a commercial name of 175 MBS3A. This multi-bonded airlaid absorbent (MBAL) core contains superabsorbent polymer powder and is made by Georgia-Pacific in Steinfurt, Germany. Three samples of the same structure were prepared. The average thickness of this structure was 2.0 mm and the average basis weight was 188 gsm.

Structure 13A was tested for liquid acquisition characteristics according to the method described in Example 3, except Structure 13A did not need to be placed on any absorbent core for liquid acquisition time measurements because Structure 13A contained superabsorbent polymer powder. FIG. 27 summarizes the average acquisition times of Structure 13A for each of the three insults. For comparison the results for the control absorbent core 175 MBS3A without any additional top layer are shown in FIG. 27 as well.

### Example 14

### Nonwoven Structure Containing Superabsorbent Polymer Powder

The present Example provides for an experimental structure (Structure 14A) containing superabsorbent polymer powder. The structure was made using a pilot-scale drum-forming airlaid line.

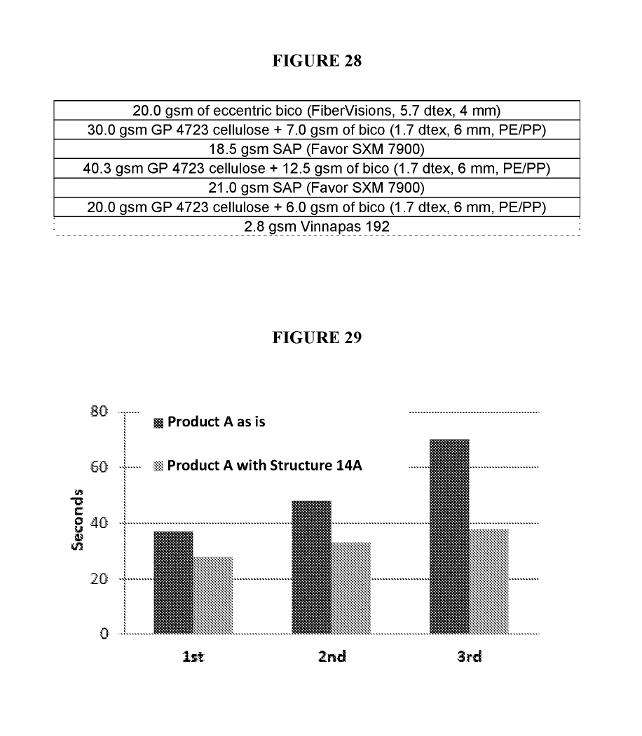

FIG. 28 depicts Structure 14A. During the process of making nonwoven samples using airlaid equipment, the total basis weight of the product can fluctuate such that parts of the product have higher or lower total basis weight compared to the target basis weight. Therefore, although FIG. 28 depicts the target basis weight, the samples of Structure 14A exhibited certain variations in the basis weight.

Structure 14A was tested for liquid acquisition performance. The tests were conducted by the SGS Courtray lab, 2 Rue Charles Montsarrat, 59500 Douai, France, using a modified SGS standard procedure POA/DF4. Rather than a plastic cylinder, a metal cylinder was used to exert a certain pressure on the tested absorbent product in order to better mimic real use conditions (e.g., when a user sits on an absorbent product). The metal cylinder was used to deliver 4 mL of the liquid to the structure at a rate of 10 mL/min. The metal cylinder had an inner diameter of 3.8 cm. The weight of the metal cylinder was 350 grams.

The structure was also tested for so-called humidity sensation, an alternative to the method of testing rewet characteristics described in previous Examples. The tests were conducted by the SGS Courtray lab, 2 Rue Charles Montsarrat, 59500 Douai, France, using SGS standard procedure POA/DF7-8. The humidity sensation test was performed using mannequins in standing and sitting positions. A collagen-based material was used to collect remaining liquid from the topsheet of the tested absorbent product. Using a collagen-based material rather than a cellulose-based material can better mimic the real use of a personal care product, because the main component of human skin is collagenous tissue.

Two commercial products, A and B, were used as controls in both tests. Product A was a sanitary napkin made by a major brand manufacturer and its absorbent system was composed of a topsheet, an acquisition layer containing a spunlace synthetic material and an airlaid absorbent core. Product B was a private label sanitary napkin and its absorbent system was composed of a topsheet, an acquisition layer containing a latex-bonded airlaid nonwoven and an absorbent core.

For each test, a given control product (Product A or Product B) was tested as is for liquid acquisition performance and humidity sensation characteristics. Then, a new sample of the same control product was prepared by removing the acquisition layer with the absorbent core and then replacing the layers into the product. Then, the reassembled product was tested. The results of the original product were comparable to the results of the reassembled product. Therefore, the disassembling and reassembling procedures did not have a significant impact on the performance of the original product and a reassembled product containing Structure 14A can be compared to the original control products.

To evaluate the effect of Structure 14A on the performance of Products A and B, the original acquisition layers and absorbent cores were replaced with Structure 14A. The sample of Structure 14A used in the series of tests with Product A had a basis weight of about 195 gsm. By comparison, the basis weight of the acquisition layer of Product A was 55 gsm and the basis weight of its absorbent core was about 190 gsm. The sample of Structure 14A used in the series of tests with Product B had a basis weight of about 180 gsm. By comparison, the basis weight of the acquisition layer of Product B was 60 gsm and the basis weight of its absorbent core was about 277 gsm. Therefore, without including the topsheets, the total basis weights of the absorbent systems (i.e., the acquisition layers and absorbent cores) of Products A and B were significantly higher that the basis weight of Structure 14A.

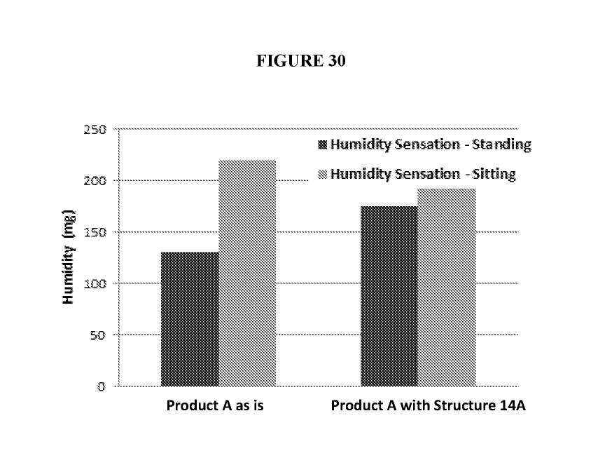

FIG. 29 illustrates the acquisition times of original Product A compared to Product A containing Structure 14A for each of the three insults. The sample containing Structure 14A showed improved acquisition times. FIG. 30 illustrates the performance of each sample in the humidity sensation test. The humidity sensation is provided as a weight (mg). The sample containing Structure 14A showed decreased humidity sensation in the sitting position compared to original Product A.

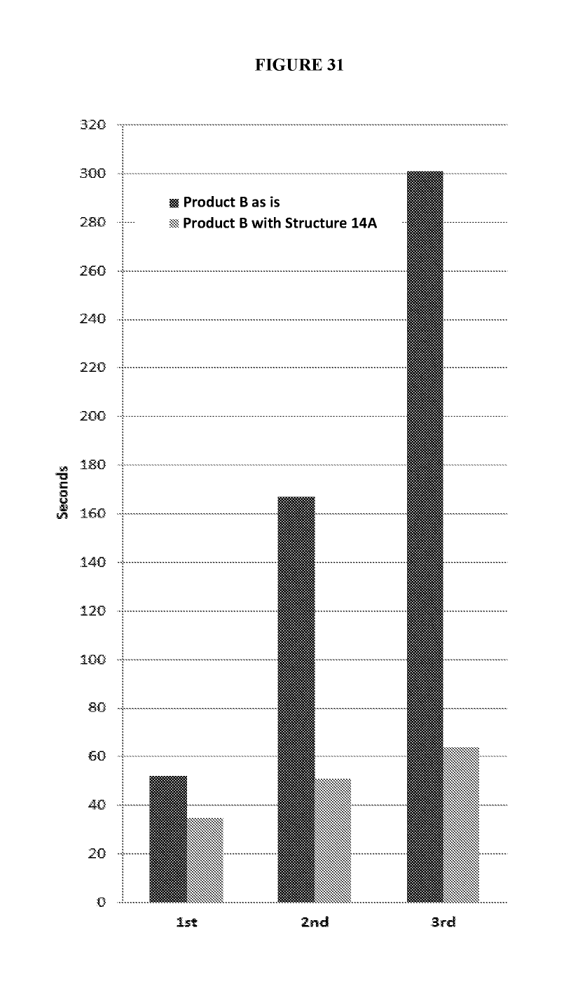

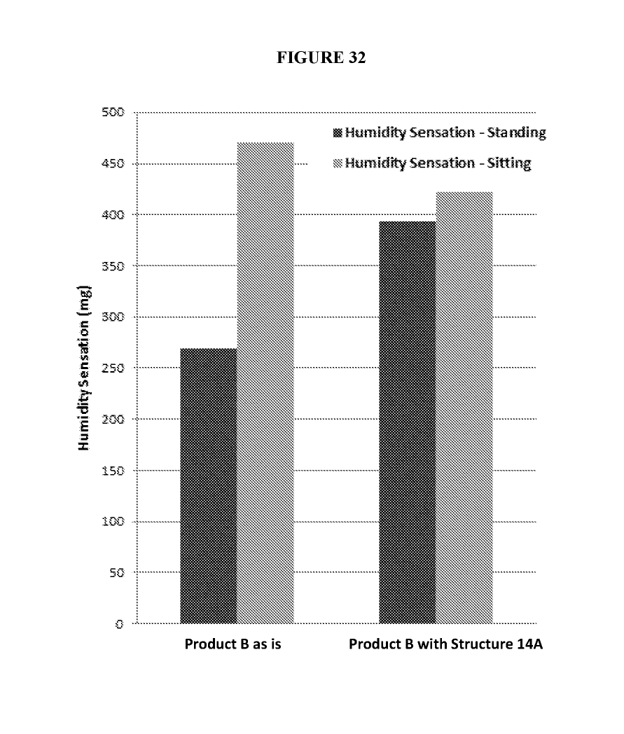

Similarly, FIG. 31 illustrates the acquisition times of original Product B compared to Product B containing Structure 14A for each of the three insults. The sample containing Structure 14A showed improved acquisition times. FIG. 32 illustrates the performance of each sample in the humidity sensation test. The humidity sensation is provided as a weight (mg). The sample containing Structure 14A showed decreased humidity sensation in the sitting position compared to original Product B.

These data suggest that Structure 14A has improved liquid acquisition performance compared to the incumbent acquisition layer/absorbent core system in both commercially available Products A and B. Furthermore, Structure 14A showed improved performance in the humidity sensation test for the more demanding sitting position.

### Example 15

### Nonwoven Structures Containing Superabsorbent Polymer Powder and Acquisition Layers

Raw materials used in this experiment included GP 4723 cellulose softwood pulp (Georgia-Pacific), eccentric bicomponent fibers, 4 mm long, 5.7 dtex (FiberVisions), and superabsorbent polymer powder (SAP) (BASF HySorb FEM 33 N).

The sheets were dry-formed on a lab-scale padformer. This procedure requires that a cellulose tissue carrier be placed on the screen of the equipment to lay the components of the formed sheets. Later, in each case, this tissue was removed from the formed structure. This was done before applying moisture and heat to bond the formed structures.

The basic absorbent core (Core) was built with five layers. The bottom layer was the GP cellulose softwood pulp in an amount of 26% of the total weight of the core, the second layer was formed with the BASF SAP in an amount of 11% of the total weight of the core, the third layer was the GP cellulose pulp in an amount of 26% of the total weight of the core, the fourth layer was the BASF SAP in an amount of 11% of the total weight of the core and the fifth, top layer was the GP cellulose pulp in an amount of 26% of the total weight of the core. The average total basis weight of the core was 153 gsm, based on three measurements. The average thickness of the core was 1.73 mm, based on three measurements.

Structure 15A was formed in such a way that it contained the same layers as the Core and they were positioned in the same order from the bottom to the top. In addition to these layers one more layer was formed on the top of the structure, which was composed of the FiberVisions bicomponent fibers in an amount of 5.4% of the total weight of Sample 15A. The average total basis weight of Sample 15A was 165 gsm, based on three measurements. The average thickness of Sample 15A was 2.10 mm, based on three measurements.

Structures 15B and 15C were similar to Structure 15A except for the amounts of the FiberVisions bicomponent fibers used in the very top layer. These amounts were, respectively, 10.3% and 15.4% of the total basis weights of Structures 15B and 15C. The average total basis weights of Structure 15B and 15C were, respectively, 175 gsm, based on three measurements, and 179 gsm, based on two measurements. The average thicknesses of Structures 15B and 15C were, respectively, 2.41 mm, based on three measurements, and 2.42 mm, based on two measurements.

Structures 15A, 15B and 15C were designed as unitary structures containing synthetic top layers which were added to improve the liquid acquisition performance of these structures.

The Core and Structures 15A, 15B and 15C were placed in each case on a nylon screen and covered with another nylon screen and three pieces of blotter paper. The paper was wetted with water and the entire configuration was nipped pressed one time using the couch press and 1 bar of pressure. The wet structure was removed from the screen and placed on the oven rack. The structures were then placed in a lab thru-air oven at 150° C. and dried for 15 minutes. After that each dry sample was cut to smaller pieces and heated at 105° C. for 15 minutes.

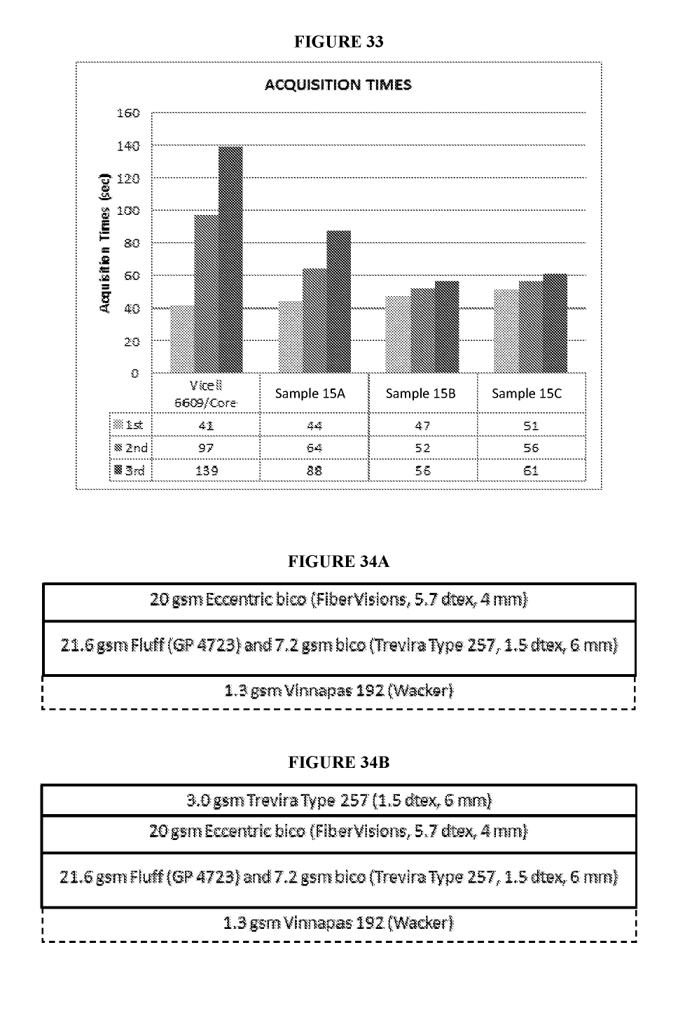

Structures 15A, 15B and 15C were tested for liquid acquisition characteristics according to the method described in Example 3, except they did not need to be placed on any absorbent core because these structures contained superabsorbent polymer powder. FIG. 33 summarizes the average acquisition times of Structures 15A, 15B and 15C for each of the three insults. For comparison the same test was conducted for the Core described in this Example, upon which a commercial acquisition layer was placed. This layer was a Georgia-Pacific commercial product, Vicell 6609. The results are shown in FIG. 33.

### Example 16

### Nonwoven Structures for Liquid Acquisition in Baby Diapers and Adult Incontinence Articles

The present Example provides for experimental structures composed of a bonded synthetic fiber top layer and a bottom layer containing bonded cellulose fibers. The fibers of the top layer can be for example bicomponent fibers such as fibers having the thickness of 5.7 dtex and length of 4 mm, made by FiberVisions, or polyester fibers bonded with bicomponent fibers or a liquid binder, and cured. The bottom layer can be composed of cellulose fibers, for instance cellulose pulp, which can be bonded with bicomponent fibers, liquid binder or with hydrogen bonds. The structures of Example 16 have basis weights in the range of 40 gsm to 200 gsm.

### Example 17

### Nonwoven Structures for Liquid Acquisition

The present Example provides for two experimental airlaid absorbent nonwoven structures (Structures 17A and 17B). The nonwoven structures were made using a pilot-scale drum-forming airlaid line.

FIG. 34A depicts Structure 17A. The first layer of Structure 17A was composed of 20 gsm of eccentric bicomponent fibers (FiberVisions, 5.7 dtex, 4 mm) and the second layer was composed of 21.6 gsm of cellulose fluff (GP 4723, fully treated pulp made by Georgia-Pacific) and 7.2 gsm of bicomponent fibers (Trevira Type 257, 1.5 dtex, 6 mm). FIG. 34B depicts Structure 17B. Structure 17B was composed of the two layers of Structure 17A, but additionally contained a top layer adjacent to the first layer and composed of 3.0 gsm of bicomponent fibers (Trevira Type 257, 1.5 dtex, 6 mm).

Samples of Structures 17A and 17B were tested for their liquid acquisition performance and for humidity sensation using the methods described in Example 14. The tests were conducted by the SGS Courtray lab, 2 Rue Charles Montsarrat, 59500 Douai, France, using SGS standard procedures. As in Example 14, Products A and B were used as controls.

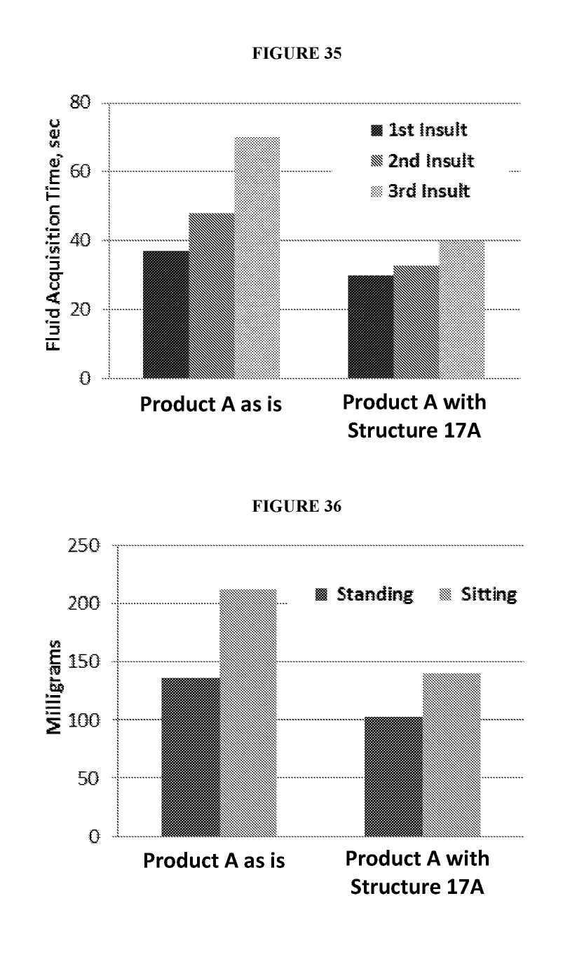

To evaluate the effect of Structure 17A on the performance of Product A, the original acquisition layer of Product A was replaced with Structure 17A for the three insults. FIG. 35 illustrates the acquisition times of original Product A compared to Product A containing Structure 17A. The sample containing Structure 17A showed improved acquisition times. FIG. 36 illustrates the performance of each sample in the humidity sensation test. The humidity sensation is provided as a weight (mg). The sample containing Structure 17A showed decreased humidity sensation in both the standing and sitting positions compared to original Product A. These data suggest that Structure 17A has improved liquid acquisition and retention performance compared to the acquisition layer of Product A.

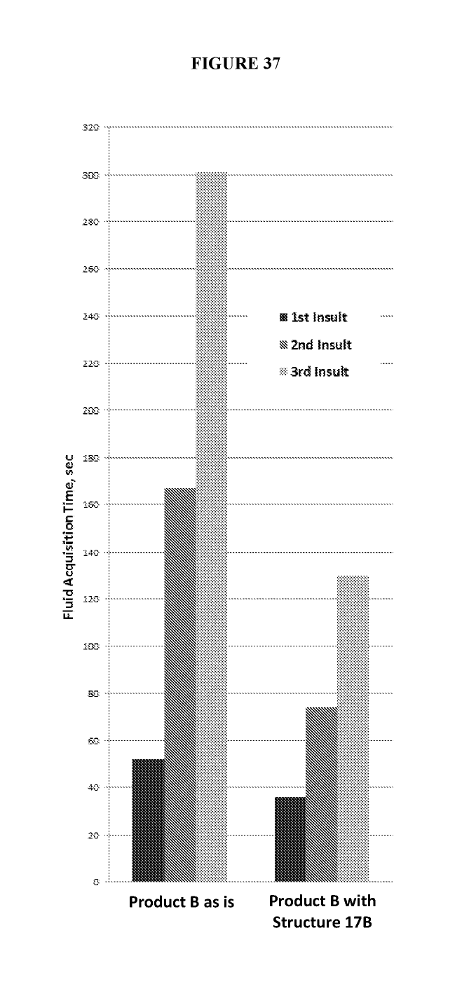

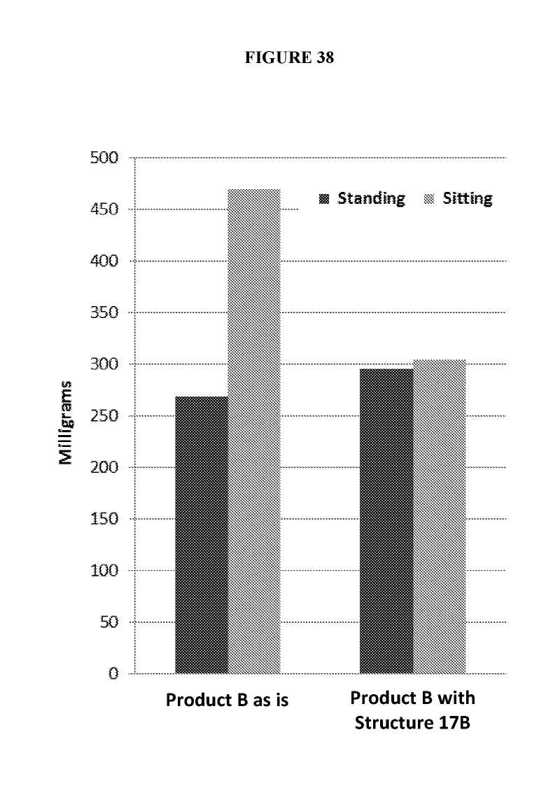

Similarly, to evaluate the effect of Structure 17B on the performance of Product B, the original acquisition layer of Product B was replaced with Structure 17B. FIG. 37 illustrates the acquisition times of original Product B compared to Product B containing Structure 17B for the three insults. The sample containing Structure 17B showed improved acquisition times. FIG. 38 illustrates the performance of each sample in the humidity sensation test. The humidity sensation is provided as a weight (mg). The sample containing Structure 17B showed decreased humidity sensation in the more demanding sitting position compared to original Product B. These data suggest that Structure 17B has improved liquid acquisition and retention performance compared to the acquisition layer of Product B.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the systems and methods of the disclosed subject matter without departing from the spirit or scope of the disclosed subject matter. Thus, it is intended that the disclosed subject matter include modifications and variations that are within the scope of the appended claims and their equivalents.

Various patents and patent applications are cited herein, the contents of which are hereby incorporated by reference herein in their entireties.

1.-20. (canceled)

21. A multi-layer nonwoven acquisition material, comprising:
a first outer layer comprising synthetic fibers and having a basis weight of from about 10 gsm to about 50 gsm; and
a second outer layer comprising cellulose fibers and having a basis weight of from about 10 gsm to about 100 gsm;
wherein the multi-layer nonwoven acquisition material has a caliper from about 0.5 mm to about 4 mm and a basis weight of from about 10 gsm to about 200 gsm

22. The multi-layer nonwoven acquisition material of claim 21, wherein the second outer layer further comprises a binder.

23. The multi-layer nonwoven acquisition material of claim 21, wherein the synthetic fibers comprise polyethylene terephthalate fibers.

24. The multi-layer nonwoven acquisition material of claim 21, wherein the synthetic fibers comprise bicomponent fibers.

25. The multi-layer nonwoven acquisition material of claim 21, wherein the second outer layer comprises cellulose tissue.

26. The multi-layer nonwoven acquisition material of claim 21, further comprising a first intermediate layer, adjacent to the first outer layer, comprising bicomponent fibers.

27. The multi-layer nonwoven acquisition material of claim 26, further comprising a second intermediate layer, adjacent to the first intermediate layer, comprising bicomponent fibers.

28. The multi-layer nonwoven acquisition material of claim 27, wherein the second intermediate layer further comprises cellulose fibers.

29. The multi-layer nonwoven acquisition material of claim 21, wherein multi-layer nonwoven acquisition material has a tensile strength at peak load of greater than about 400 G/in.

30. A multi-layer nonwoven material, comprising:
the multi-layer nonwoven acquisition material of claim 21; and
an absorbent core;
wherein the multi-layer nonwoven material has a caliper from about 1 mm to about 8 mm and a basis weight of from about 100 gsm to about 600 gsm.

31. The multi-layer nonwoven material of claim 30, wherein the absorbent core comprises:
a first layer comprising cellulose fibers;
a second layer, adjacent to the first layer, comprising SAP;
a third layer, adjacent to the second layer, comprising cellulose fibers;
a fourth layer, adjacent to the third layer, comprising SAP; and
a fifth layer, adjacent to the fourth layer, comprising cellulose fibers.

32. The multi-layer nonwoven material of claim 31, wherein at least one of the first layer, third layer, and fifth layer further comprise bicomponent fibers.

33. An absorbent composite comprising the multi-layer nonwoven acquisition material of claim 21.

34. A multi-layer nonwoven acquisition material, comprising:
a first outer layer comprising synthetic fibers and having a basis weight of from about 10 gsm to about 50 gsm;
a first intermediate layer, adjacent to the first outer layer, comprising bicomponent fibers;
a second intermediate layer, adjacent to the first intermediate layer, comprising cellulose fibers and bicomponent fibers; and
a second outer layer, adjacent to the second intermediate layer, comprising cellulose fibers and a binder and having a basis weight of from about 10 gsm to about 100 gsm;
wherein the multi-layer nonwoven acquisition material has a caliper of from about 0.5 mm to about 4 mm and a basis weight of from about 10 gsm to about 200 gsm.

35. A multi-layer nonwoven acquisition material, comprising:
a first outer layer comprising bicomponent fibers and having a basis weight of from about 10 gsm to about 50 gsm;
a first intermediate layer, adjacent to the first outer layer, comprising bicomponent fibers; and
a second intermediate layer, adjacent to the first intermediate layer, comprising bicomponent fibers; and
a second outer layer, adjacent to the second intermediate layer, comprising cellulose fibers and a binder and having a basis weight of from about 10 gsm to about 100 gsm;
wherein the multi-layer nonwoven acquisition material has a caliper of from about 0.5 mm to about 4 mm and a basis weight of from about 10 gsm to about 200 gsm.

36. A multi-layer nonwoven acquisition material, comprising:
a first outer layer comprising synthetic fibers and having a basis weight of from about 10 gsm to about 50 gsm; and
a second outer layer comprising synthetic filaments;
wherein the multi-layer nonwoven acquisition material has a caliper from about 0.5 mm to about 4 mm and a basis weight of from about 10 gsm to about 200 gsm.

37. The multi-layer nonwoven acquisition material of claim 36, wherein the first outer layer further comprises a binder.

38. The multi-layer nonwoven acquisition material of claim 36, wherein the synthetic fibers comprise bicomponent fibers.

39. The multi-layer nonwoven acquisition material of claim 36, further comprising a first intermediate layer, adjacent to the first outer layer, comprising bicomponent fibers.

40. The multi-layer nonwoven acquisition material of claim 39, further comprising a second intermediate layer, adjacent to the first intermediate layer, comprising bicomponent fibers.

41. The multi-layer nonwoven acquisition material of claim 36, wherein multi-layer nonwoven acquisition material has a tensile strength at peak load of greater than about 400 G/in.

42. A multi-layer nonwoven material, comprising:
the multi-layer nonwoven acquisition material of claim 36; and
an absorbent core;
wherein the multi-layer nonwoven material has a caliper from about 1 mm to about 8 mm and a basis weight of from about 100 gsm to about 600 gsm.

43. The multi-layer nonwoven material of claim 42, wherein the absorbent core comprises:
a first layer comprising cellulose fibers;
a second layer, adjacent to the first layer, comprising SAP;
a third layer, adjacent to the second layer, comprising cellulose fibers;
a fourth layer, adjacent to the third layer, comprising SAP; and
a fifth layer, adjacent to the fourth layer, comprising cellulose fibers.

44. The multi-layer nonwoven material of claim 43, wherein at least one of the first layer, third layer, and fifth layer further comprise bicomponent fibers.

45. An absorbent composite comprising the multi-layer nonwoven acquisition material of claim 36.
